(19) United States
(12) Patent Application Publication
(10) Pub. No.: US 2023/0250433 A1
(43) Pub. Date: Aug. 10, 2023

(54) METHODS AND COMPOSITIONS FOR TREATMENT OF APC-DEFICIENT CANCER

(71) Applicant: Board of Regents, The University of Texas System, Austin, TX (US)

(72) Inventors: Rumi LEE, Houston, TX (US); Ronald A DEPINHO, Houston, TX (US)

(73) Assignee: Board of Regents, The University of Texas System, Austin, TX (US)

(21) Appl. No.: 17/905,124

(22) PCT Filed: Feb. 26, 2021
(86) PCT No.: PCT/US2021/019989

§ 371 Date: Aug. 26, 2022

Related U.S. Application Data
(60) Provisional application No. 62/982,561, filed on Feb. 27, 2020.

(51) Int. Cl. C12N 15/113; A61K 31/4439; A61K 31/496; A61K 31/713; C12Q 1/6886; A61P 35/00

(52) U.S. Cl. CPC C12N 15/1137; A61K 31/4439; A61K 31/496; A61K 31/713; C12Q 1/6886; A61P 35/00; C12Q 2600/158

(57) ABSTRACT

The disclosure provides methods and compositions for treating subject having cancers with abnormal WNT signaling, such as APC-deficient cancers. Accordingly, aspects of the disclosure relate to methods for treating APC-deficient cancers comprising administering an inhibitor of TDO2 or of a cytokine activated by TDO2. Additional aspects relate to methods for identifying a cancer as being sensitive to TDO2 inhibition comprising detecting a deficiency in an APC gene and/or measuring an expression level of TDO2.

### CROSS-REFERENCE

This application claims benefit of priority of U.S. Provisional Application No. 62/982,561, filed Feb. 27, 2020, which is hereby incorporated by reference in its entirety.

This invention was made with government support under R01CA231360 and 5T32CA186892-04 awarded by the National Institutes of Health. The government has certain rights in the invention.

### SEQUENCE LISTING

The instant application contains a Sequence Listing which has been submitted in ASCII format and is hereby incorporated by reference in its entirety. Said ASCII copy, created on Feb. 23, 2021, is named UTSCP1205WO—SeqListing.txt and is 3,899 bytes in size.

### BACKGROUND

### I. Field of the Invention

Aspects of the invention relate to at least the fields of molecular biology, immunology, and oncology.

### II. Background

Colorectal cancer (CRC) is the second leading cause of cancer-related death in developed countries and responsible for more than 600,000 deaths globally each year. The evolution of CRC from adenoma to adenocarcinoma and ultimately invasive and metastatic disease is governed by the acquisition of signature genetic alterations, most prominently inactivation of adenomatous polyposis coli (APC) and p53 tumor suppressors and activation of the KRAS oncogene (Orchard et al., 2013). APC loss also occurs frequently across many other cancer types (Fang et al., 2002; Furuuchi et al., 2000; Horii et al., 1992; Ohgaki et al., 2004). With respect to the APC pathway, mutated APC proteins lose the ability to activate glycogen synthase kinase 3β (GSK3β) which in turn phosphorylates N-terminal serine/threonine residues of β-catenin, mediating β-catenin degradation through ubiquitination. Thus, APC deficiency results in the accumulation of β-catenin which then moves to the nucleus to bind and de-repress the T-cell factor/lymphoid enhancer-binding factor (TCF/LEF) transcription factor complex, enabling activation of the canonical WNT signaling network.

Despite its importance in cancer, the therapeutic targeting of this APC signaling cascade remains an elusive goal for cancer therapy. Currently, agents targeting the WNT pathway include inhibitors of WNT ligands, b-catenin degrading complex, TCF/LEF, and Notch and Sonic Hedgehog signaling that crosstalk with WNT. To date, these WNT targeting programs have yet to produce meaningful clinical results. There remains a need for orthogonal strategies to target APC-deficient colorectal cancer (CRC) and other APC-deficient tumor types.

### SUMMARY

The disclosure provides for methods and compositions suitable for treating a subject having an APC-deficient cancer. Accordingly, aspects of the disclosure are directed to methods for treating a subject with an APC-deficient cancer comprising providing to the subject an inhibitor of TDO2 or of a cytokine activated by TDO2 activity. Further aspects are directed to methods for treating a subject for cancer comprising identifying the cancer as having an APC deficiency and providing to the subject an inhibitor of TDO2 or a cytokine activated by TDO2 activity. Additional aspects comprise methods for identifying a cancer as being sensitive to TDO2 inhibition comprising detecting a deficiency in an APC gene and/or an increase in expression of TDO2 in the cancer. Yet further aspects relate to methods for treating a subject for a cancer having constitutively active WNT signaling comprising providing to the subject an inhibitor of TDO2 or of a cytokine activated by TDO2 activity.

Various embodiments of the disclosure include methods for treating a subject with an APC-deficient cancer, methods for treating a subject with a cancer determined to have an APC mutation, methods for treating a subject with APC-deficient CRC, methods for identifying a cancer as being susceptible to TDO2 inhibition, methods for identifying a cancer as being susceptible to inhibition of a cytokine activated by TDO2 activity, methods for diagnosing a patient, methods for prognosing a patient, methods for detecting cancer cells, compositions for treating APC-deficient cancer, and methods for treating a subject with a cancer having constitutively active WNT signaling.

Methods of the disclosure can include at least 1, 2, 3, 4, 5, 6, 7, 8, 9, 10, or more of the following steps: providing a TDO2 inhibitor, providing an inhibitor of a cytokine, identifying a cancer as having an APC deficiency, providing a pharmaceutical composition to a subject, sequencing a nucleic acid from cancer cells, identifying a mutation in a nucleic acid from cancer cells, comparing a nucleic acid expression level to a control or reference value, analyzing a methylation status of a nucleic acid from cancer cells, obtaining a biological sample, isolating cancer cells from a subject, treating a subject with a cancer therapy, treating a subject with immunotherapy, detecting an increased expression of a gene, determining an outcome of a treatment, identifying a cancer as being sensitive to TDO2 inhibition, identifying a cancer as being sensitive to inhibition of a cytokine activated by TDO2 activity, inhibiting TDO2, inhibiting CXCL5, and inhibiting CXCL1/2. In some embodiments, any one or more steps are contemplated as being excluded from the disclosed methods.

In some embodiments, disclosed herein is a method for treating a subject with a cancer determined to have an adenomatous polyposis coli (APC) mutation, the method comprising providing to the subject a pharmaceutical composition comprising an effective amount of an inhibitor of (a) tryptophan 2,3-dioxygenase (TDO2) or (b) a cytokine activated by TDO2 activity. In some embodiments, disclosed herein is a method for treating a subject for an APC-deficient cancer, the method comprising providing to the subject a pharmaceutical composition comprising an effective amount of an inhibitor of (a) TDO2 or (b) a cytokine activated by TDO2 activity. In some embodiments, disclosed herein is a method for treating a subject for cancer, the method comprising (a) identifying the cancer as having an APC deficiency and (b) providing to the subject a pharmaceutical composition comprising an effective amount of an inhibitor of (i) TDO2 or (ii) a cytokine activated by TDO2 activity. In some embodiments, disclosed herein is a method for treating cancer in a subject comprising determining whether the cancer has an APC mutation and (a) if the cancer has an APC mutation, providing to the subject an effective amount of an inhibitor of (ii) TDO2 or (ii) a cytokine activated by TDO2 activity and (b) if the cancer does not have an APC mutation, providing to the subject an effective amount of an alternate therapy. An alternate therapy may be, for example, radiotherapy, chemotherapy, or surgery.

In some embodiments, an inhibitor is an inhibitor of TDO2. In some embodiments, the inhibitor inhibits the expression of TDO2 in the cancer. In some embodiments, the inhibitor inhibits the activity of TDO2 in the cancer. In some embodiments, the inhibitor is an siRNA, an shRNA, an antisense oligonucleotide, a small molecule inhibitor, an antibody, or an antibody-like molecule. In some embodiments, the inhibitor is an siRNA, shRNA, or antisense oligonucleotide targeting TDO2. In some embodiments, the inhibitor is PF06845102/EOS200809, 680C91, LM10, HTI-1090, DN1406131, RG70099, EPL-1410, CB548, CMG017, or a derivative thereof. In some embodiments, the inhibitor is PF06845102/EOS200809. In some embodiments, the inhibitor is 680C91. In some embodiments, the pharmaceutical composition is administered intravenously, intramuscularly, intraperitoneally, intracerobrospinally, subcutaneously, intra-articularly, intrasynovially, intrathecally, orally, topically, through inhalation, or through a combination of two or more routes of administration.

In some embodiments, the inhibitor is an inhibitor of a cytokine activated by TDO2 activity. In some embodiments, the cytokine is CXCL5, CXCL7, CSF3, CXCR2, CXCL2, CXCL10, CCL2, or CXCL1. In some embodiments, the cytokine is CXCL5, CXCL7, or CSF3. In some embodiments, the cytokine is CXCL5. In some embodiments, the inhibitor is a CXCL1/2 inhibitor. In some embodiments, the inhibitor is a CXCL5 inhibitor. In some embodiments, the inhibitor is an antibody or antibody-like molecule targeting CXCR5. In some embodiments, the inhibitor is an siRNA, shRNA, or an antisense oligonucleotide targeting CXCL5.

Disclosed herein, in some aspects, is a method for identifying a cancer as being sensitive to TDO2 inhibition, the method comprising (a) obtaining cancer cells from a biological sample from a subject; (b) detecting a deficiency in an APC gene in the cancer cells; and (c) identifying the cancer as being sensitive to TDO2 inhibition based on (b). Also disclosed, in some embodiments, is a method for determining whether a biological sample comprises cancer cells sensitive to TDO2 inhibition, the method comprising (a) measuring a presence or absence of an APC deficiency in the biological sample; (b) determining that the biological sample comprises cancer cells sensitive to TDO2 inhibition if the biological sample comprises an APC deficiency; and (c) determining that the biological sample does not comprise cancer cells sensitive to TDO2 inhibition if the biological sample does not comprise an APC deficiency.

In some embodiments, the method further comprises detecting an increased expression or activity of TDO2 in the cancer cells relative to cells from a non-cancerous biological sample. In some embodiments, the method further comprises measuring an expression or activity level of TDO2 in the biological sample. In some embodiments, the method further comprises determining that the biological sample does not comprise cancer cells sensitive to TDO2 inhibition if the TDO2 expression or activity level in the biological sample is not significantly different from the expression or activity level in a non-cancerous biological sample. In some embodiments, an APC deficiency is an APC mutation. In some embodiments, the APC deficiency is a repression of APC expression. In some embodiments, the repression is an epigenetic repression.

In some embodiments, the cancer has an increased expression of TDO2 relative to a control or reference sample, which may be a biological sample from a healthy subject. In some embodiments, the cancer comprises constitutively active WNT signaling. In some embodiments, the cancer is colorectal cancer, breast cancer, prostate cancer, lung cancer, head and neck squamous cell carcinoma, or sarcoma. In some embodiments, the cancer is colorectal cancer. In some embodiments, the cancer is a cancer of a given diagnostic stage. In some embodiments, the cancer is a stage I, stage IIa, stage IIb, stage IIc, stage IIIa, stage IIIb, stage IIIc, stage IVa, or stage IVb cancer. In some embodiments, the cancer is recurrent cancer. In some embodiments, the subject was previously treated for the cancer. In some embodiments, the subject was not previously treated for the cancer. In some embodiments, the cancer was determined to be resistant to a previous treatment.

In some embodiments, the disclosed methods further comprise providing to the subject a cancer immunotherapy. A cancer immunotherapy may be, for example, an antibody therapy or a cellular therapy. In some embodiments, the cancer immunotherapy is a checkpoint inhibitor therapy. In some embodiments, the method further comprises providing to the subject an additional therapy wherein the additional therapy is chemotherapy, radiation therapy, surgery, or a combination thereof. In some embodiments, the method does nor comprise any step of providing to the subject chemotherapy, radiation therapy, or surgery. In some embodiments, the method comprises reducing a number of tumor associated macrophages in the subject.

In some embodiments, a biological sample is a blood or serum sample. In some embodiments, cancer cells are colorectal cancer cells. In some embodiments, a biological sample is a tissue sample adjacent to a surgical site of a colorectal cancer patient. In some embodiments, a non-cancerous biological sample is normal mucosal tissue.

In some embodiments, the disclosed methods further comprise providing to the subject an inhibitor of indoleamine 2,3-dioxygenase 1 (IDO1) or indoleamine 2,3-dioxygenase 2 (IDO2). In some embodiments, the method does not comprise providing to the subject an inhibitor of IDO1 or IDO2.

Aspects of the disclosure are directed to a method for treating a subject for a cancer determined to have constitutively active WNT signaling, the method comprising providing to the subject a pharmaceutical composition comprising an effective amount of an inhibitor of (a) TDO2 or (b) a cytokine activated by TDO2 activity. In some embodiments, the constitutively active WNT signaling comprises an APC-deficiency. In some embodiments, the constitutively active WNT signaling does not comprise an APC-deficiency. In some embodiments, the cancer comprises a constitutively active β-catenin protein.

Additional aspects are directed to use of (a) an inhibitor of (i) TDO2 or (ii) a cytokine activated by TDO2 activity and (b) a pharmaceutical acceptable carrier in the treatment of an APC-mutant cancer in a subject or in the manufacture of a medicament for treating an APC-mutant cancer in a subject.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or. Is is specifically contemplated that A, B, or C may be specifically excluded from an embodiment.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F show results from studies revealing TDO as a synthetic essential gene for mutant APC gene in CRC. FIG. 1A shows mutual exclusive patterns of TDO2 and APC/CTNNB1 in TCGA database of multiple cancer types. The percentages of alteration in each gene are indicated. FIG. 1B shows Venn diagram analysis using two different datasets identified three potential SE genes. FIG. 1C shows that TDO2 mRNA expression is significantly correlated with expression of WNT pathway signature genes in TCGA CRC (COAD+READ, Provisional) patients (n=433). ***P<0.001. FIG. 1D shows a representation of immunohistochemistry (IHC) staining for TDO2 in human CRC tumors with negative (n=34) and positive nuclear β-catenin (n=47). Scale bars, ×10 (200 μm) and X40 (50 μm). FIG. 1E shows that CRC tumors with nuclear β-catenin showed higher TDO2 expression (TDO2 staining score 0-3). Pearson Correlation Coefficient=42.342, ****P<0.0001. Chi-squared test. FIG. 1F shows IHC analysis of polyps from APCmin mice and tumors from iKAP mouse models, revealing increased nuclear β-catenin, Ki67, and TDO2 compared to normal colon tissue. Representative images of triplicate studies are shown. Scale bar, 100 μm.

FIGS. 2A-2J show results from studies demonstrating a correlation between TDO2 expression and WNT activation in CRC. FIGS. 2A and 2B show mRNA expression of PPAT and AP3D1 in CCD-841-CoN and isogenic RKO cell lines. FIG. 2C shows a representation of clustering of TCGA COAD and READ dataset based on the expression of WNT signature genes (Van der Flier et al., 2007). APC mutation status is shown. FIG. 2D shows a representation of clustering of TCGA COAD and READ dataset based on the expression of hallmark WNT gene sets (HALLMARK_WNT_BETA_CATENIN_SIGNALING). FIG. 2E shows that expression of hallmark WNT genes are correlated with TDO2 expression in TCGA CRC (COAD+READ, Provisional) patient samples (n=433). ****P<0.0001. FIG. 2F shows that PPAT mRNA expression is reversely correlated with expression of WNT signature genes in TCGA CRC (COAD+READ, Provisional) patients (n=433). **P<0.01. FIG. 2G shows mutual exclusive patterns of IDO1/2 to APC and CTNNB1 in TCGA CRC (COAD+READ, Provisional) dataset (n=220). FIG. 2H shows that IDO1 mRNA expression is not correlated with expression of WNT signature genes in TCGA CRC (COAD+READ, Provisional) patients (n=433). n.s. P>0.05, **P<0.01. FIG. 2I shows that IDO2 mRNA expression shows correlation with expression of WNT signature genes in TCGA CRC (COAD+READ, Provisional) patients (n=433). FIG. 2J shows Venn diagrams representative of analysis to identify SE genes for APC mutations in CRC. Two separate analyses using different cutoffs are shown. *Left P value—P value for genes that are essential for β-catenin active cancer cell lines.

FIGS. 3A-3I show results from studies demonstrating that TCF4/TCF7L2 mediates upregulation of TDO2 in APC-mutated CRC cells. FIG. 3A shows immunoblots for TDO2 and β-catenin in CRC cell lines RKO (human) and MC38 (Mouse) with their isogenic APC-KO counterparts. FIG. 3B shows RT-qPCR results demonstrating that APC deleted RKO and MC38 cell lines exhibit increased TDO2 mRNA expression. ****P<0.0001. FIG. 3C shows the DNA sequence binding motif for transcription factor TCF4/TCF7L2. FIG. 3D shows that promoter regions of human and mouse TDO2 gene harbor TCF4 binding motifs near transcription starting site. The motif sequence is conserved in human and mouse genes. FIG. 3E shows that ChIP-seq in APC-WT and APC-KO MC38 cells showed binding peaks for TCF4 on the promoters of TDO2 gene. FIG. 3F shows that CHIP-PCR using TCF4 antibody showed enriched binding to the promoter regions of TDO2 gene in DLD-1 cells. GAPDH as a negative control and MYC and AXIN2 as positive controls. FIG. 3G shows results from a luciferase activity of hTDO2 promoter in HEK 293T cells with constitutive active form of β-catenin (Δ90) when co-transfected with dominant negative (DN) TCF4. n=3 biological replicates. n.s.P>0.05, *P≤0.05, **P<0.01. FIG. 3H shows a luciferase activity of hTDO2 promoter and TCF4 binding motif-mutated hTDO2 promoter in HEK 293T cells with constitutive active form of β-catenin (Δ90) and dominant negative (DN) TCF4. n=3 biological replicates. n.s.P>0.05, **P<0.01. FIG. 3I shows immunoblotting of APC and TDO2 in uncloned MC38 cells, APC-positive clones, and APC-deleted clones. FIG. 3J shows immunoblotting of TDO2 in MC38 cells supplemented with 0, 50 and 100 ng of recombinant mouse WNT3a proteins for 36 hrs. FIGS. 3K-3M show immunoblot for TDO2 and β-catenin in DLD-1 cells (FIG. 3K), Caco-2 cells (FIG. 3L), and HCT-15 cells (FIG. 3M) treated with XAV-939 in a dose-dependent manner.

FIGS. 4A-4P show results from studies demonstrating that TDO2 is essential for survival in APC-mutated CRC cells. FIG. 4A shows RT-qPCR demonstrating TDO2 shRNA knockdown efficiency in APC-WT and APC-KO MC38 cell lines. n=3 biological replicates. FIG. 4B shows representative images of a colony formation assay of APC-WT and APC-KO MC38 cell lines expressing shTDO2. n=3 biological replicates. FIG. 4C shows representative images of crystal violet staining of APC-WT and APC-KO MC38 cell lines after treating 680C91 in a dose-dependent manner for 48 hr. n=3 biological replicates. FIG. 4D shows immunofluorescence staining of RFP-caspase-3 in APCmin colonoids infected with ishTDO2 after 96 h of dox treatment. Induction of shTDO2 is indicated by GFP (highlighted with arrows). X63 magnification. FIG. 4E shows immunoblots for TDO2 and cleaved Caspase-3 in APCmin ishTDO2 organoid cell lysates after Dox treatment for 48 h. FIG. 4F shows RT-qPCR demonstrating TDO2 inducible shRNA knockdown efficiency in APC-WT and APC-KO MC38 cell lines. n=3 biological replicates. FIG. 4G shows immunoblots of cleaved caspase-3 in APC-WT and APC-KO MC38 cell lines with ishTDO2 after doxycycline (Dox) treatment. FIG. 4H shows representative in vivo bioluminescence-based images of C57BL/6J mice at day 21 post-orthotopic injection of ishTDO2 APC-WT and APC-KO MC38 cell lines (2×10⁵ cells). Dox food was provided at day 5 after injection to induced TDO2 knockdown in vivo (n=5 per group). FIG. 4I shows total flux measurement of tumors from tumors in H. n.s.P>0.05, *P<0.05. FIG. 4J shows weights of tumors harvested from mice in FIG. 4H (n=6 per group) n.s.P>0.05, ****P<0.0001. FIG. 4K shows IHC of KI67 and caspase-3 in CRC orthotopic tumors tissues generated from samples in FIG. 4H. Scale bar, 100μm. FIG. 4L shows survival curves of C57BL/6J mice orthotopically implanted with ishTDO2 APC-WT and APC-KO MC38 cell lines (5×10⁵ cells). Dox food was applied at day 5 post-orthotopic injection to induce TDO2 knockdown in vivo (ishTDO2 APC-WT MC38, n=5, ishTDO2 APC-WT+Dox MC38 and ishTDO2 APC-KO MC38, n=6, ishTDO2 APC-KO MC38+Dox, n=7). n.s.P>0.05, **P<0.01, ***P<0.001. two-tailed t-test. FIG. 4M shows survival curves of NSG mice orthotopically implanted with ishTDO2 APC-WT and APC-KO MC38 cell lines (5×10⁵ cells). Dox food was applied at day 5 post-orthotopic injection to induce TDO2 knockdown in vivo (n=8 per group). n.s.P>0.05, **P<0.01, ***P<0.001. two-tailed t-test. FIGS. 4N and 4O show survival curves of C57BL/6J mice orthotopically implanted with APC-WT and APC-KO MC38 cell lines (5=10⁵ cells) treated with TDO2 inhibitor (FIG. 4N) or Epacadostat (FIG. 4O). TDO2 inhibitor and Epacadostat treatment (100 mg/kg) was initiated at day 5 post-injection twice a day by oral gavage. n.s.P>0.05, *P<0.05, ***P<0.001. Log-rank (Mantel-Cox) test. FIG. 4P shows immunohistochemistry staining of cleaved caspase-3, F4/80, CD163, and Ki67 in APC-WT and APC-KO MC38 tumors that were treated with TDO2 inhibitor (100 mg/kg) or Epacadostat (100 mg/kg) and harvested at end point. Scale bars, ×10 (200 μm).

FIGS. 5A-5I show results from studies demonstrating TDO2 essentiality in APC-mutated/WNT-active human CRC cell lines. FIG. 5A shows mutation status of APC and CTNNB1 in human normal and CRC cell lines used in the study. FIG. 5B shows RT-qPCR demonstrating TDO2 shRNA knockdown efficiency in isogenic APC-WT and APC-KO RKO as well as DLD-1, LS180, and HT-28 cell lines. n=3 biological replicates. FIG. 5C shows representative images of colony formation assay of human CRC cell lines expressing shTDO2. n=3 biological replicates. FIG. 5D shows representative images of crystal violet staining of APC-WT and APC-KO MC38 cell lines after treating 680C91 in a dose-dependent manner for 48 hr. n=3 biological replicates. FIG. 5E shows weights of tumors in NSG mice injected with APC-WT and APC-KO RKO cell lines expressing shTDO2 (5×10⁵ cells per injection). Tumors were harvested from at day 25 post-subcutaneous injection (n=7 per group) n.s.P>0.05, ****P<0.0001. two-tailed t-test. FIG. 5F shows measurement of subcutaneous tumor growth of TDO2 knockdown and hairpin-resistant (HR) TDO2-ORF expressing DLD-1 cell lines in nude mice (5×10⁵ cells per injection). n=5 per group. Total flux measurement of tumors is shown. *P<0.05, two-tailed t-test. FIGS. 5G and 5H show IHC of Ki67 and caspase-3 in tumors tissues generated from samples in FIG. S2E and S2F. Scale bar, 100 μm. FIG. 5I shows representation of bright field images of APCmin organoids with inducible TDO2 after Dox treatment for 96 h. Scale bar, 50 μm.

FIGS. 6A-6D show results from studies demonstrating reduced tumor growth by TDO2 depletion in WNT-dependent BRCA tumors. FIG. 6A shows RT-qPCR demonstrating TDO2 shRNA knockdown efficiency in ishTDO2 4T1 cell line. n=3 biological replicates. FIGS. 6B and 6C show representative images and measured weights of breast tumors generated by orthotopically injecting ishTDO2 4T1 cells (2×10⁴ cells per injection) into mammary fat pad with and without dox treatment. n=6 per group. ***P<0.001. two-tailed t-test. FIG. 6D shows IHC of Ki67 and caspase-3 in tumors tissues generated from samples in FIG. S3B. Scale bar, 100 μm.

FIGS. 7A-7J show results from studies demonstrating that upregulated kynurenine pathway by TDO2 activates AhR signaling in APC-mutant CRC cells. FIG. 7A shows GSEA correlation of Tryptophan metabolism and Xenobiotic metabolism with alternatively expressed genes in TDO2 depleted APC-KO MC38 cells. Normalized enrichment score (NES) and nominal P value are shown. FIG. 7B shows positive correlation of TDO2 expression to AhR and CYP1B1 expression in TCGA COAD database (n=286). The P value and R-square value are shown. FIG. 7C shows that IHC analysis of polyps from APCmin mice and tumors from iKAP mouse models showed increased AhR compared to normal colon tissue. Representative images of triplicate studies are shown. Scale bar, 100 μm. FIG. 7D shows results from an ELISA assay for kynurenine (kyn) present in culture media incubated with ishTDO2 APC-WT and APC-KO MC38 cell lines. n=4 biological replicates. **P<0.01. FIG. 7E shows RT-qPCR demonstrating TDO2 knockdown decreased the expression of AhR downstream target genes in APC-KO MC38 cell lines. n=3 biological replicates. FIG. 7F shows representative images of colony formation assay of ishTDO2 APC-KO MC38 cell lines after Dox alone, Dox/Kyn co-treatment, and shAhR expression. n=3 Biological replicates. FIG. 7G shows representative images from a colony formation assay of APC-KO MC38 cell lines after 680C91 alone and 680C91/Kyn co-treatment. n=3 Biological replicates. FIG. 7I shows immunoblots for cleaved Caspase-3 in APC-WT and APC-KO MC38 cell lysates treated with 680C91 alone and 680C91/Kyn. FIG. 7H shows immunoblots for cleaved Caspase-3 in iKAP cell lysates treated with 680C91 alone and 680C91/Kyn. FIG. 7J shows survival curves of C57BL/6J mice orthotopically implanted with shControl APC-KO MC38 cell lines (2×10⁵ cells) and two shAhR (#1 and #2) APC-KO MC38 cell lines (2×10⁵ cells). Dox food was applied at day 5 post-orthotopic injection to induce TDO2 knockdown in vivo (shControl, n=7, Dox, n=8, shAhR #1, #2, n=7). ***P<0.001. two-tailed t-test.

FIGS. 8A and 8B show results from studies demonstrating that TDO2 regulates cell survival and growth via AhR signaling in APC-mutated CRC cells. FIG. 8A shows RT-qPCR demonstrating decreased expression of AhR, CYP1B1 and CYP1A1 in shTDO2 DLD-1 cells. n=3 biological replicates. ****P<0.0001. FIG. 8B shows IHC of Ki67 and caspase-3 in tumors tissues generated from samples in FIG. 4J. Scale bar, 100 μm.

FIGS. 9A-9J show results from studies demonstrating that TDO2-AhR mediates tumor growth by regulating macrophage infiltration. FIG. 9A shows GSEA analysis (Hallmark gene sets) on genes that overlap between RNA-seq datasets of ishTDO2 APC-KO MC38 cell lines (No dox vs. 48 hr dox, n=3) and microarray datasets of xenograft tumors established with ishTDO2 APC-KO MC38 cell lines (No dox vs. dox treated, n=3). Only the genes that are upregulated in APC-KO cells were analyzed. The blue bars indicate immune response-related pathways. FIG. 9B shows GSEA correlation of TNFA signaling and inflammatory response with alternatively expressed genes in TDO2 depleted APC-KO MC38 cells. Normalized enrichment score (NES) and nominal P value are shown. FIG. 9C shows viSNE analysis of F4/80 and CD206 positive immune cells assessed by CyTOF from CRC orthotopic ishTDO2 APC-WT and APC-KO MC38 tumors. FIG. 9D shows quantification of macrophages (F4/80+) and M2 macrophages (CD206+) in tumors shown in FIG. 9C. CyTOF data, analyzed by FlowJo. Data represent mean±s.d. *P<0.05, **P<0.01. two-tailed t-test. FIG. 9E shows viSNE analysis of F4/80 and CD206 positive immune cells assessed by CyTOF from CRC orthotopic ishTDO2 CT26 tumors. FIG. 9F shows quantification of macrophages (F4/80+) and M2 macrophages (CD206+) in tumors established with ishTDO2 CT26 cell lines. CyTOF data were analyzed by FlowJo. Data represent mean±s.d. **P<0.01. two-tailed t-test. FIG. 9G shows survival curves of Balb/C mice orthotopically implanted with ishTDO2 CT26 cell lines (2×10⁵ cells). Clondronate liposomes or control encapsome liposomes were given intraperitoneally (100 μl) at day 2 post-orthotopic injection and three times a week (Encapsome, n=6, Clodronate, n=9). ***P<0.001 two-tailed t-test. FIG. 9H shows that TDO2 mRNA expression is significantly correlated with expression of total macrophage markers and M2 macrophage markers in TCGA CRC (COAD+READ, Provisional) patients (n=433). ****P<0.0001. FIG. 9I shows a representation of IHC staining for CD163 in human CRC tumors with negative (n=42) and positive nuclear β-catenin (n=50). Scale bars, −10 (200 μm) and λ40 (50 μm). FIG. 9J shows that CRC tumors with nuclear β-catenin showed higher CD163 expression. Pearson Correlation Coefficient=5.074, P=0.0243. Chi-squared test.

FIGS. 10A-10G show results from studies demonstrating that the TDO2-AhR axis mediates glycolysis in APC-mutated CRC cells. FIG. 10A shows GSEA correlation of glycolysis with altered gene expression in TDO2 depleted APC-KO MC38 cells. Normalized enrichment score (NES) and nominal P value are shown. FIG. 10B shows results from a cell viability assay of APC-WT and APC-KO MC38 cells treated with STF-31 for 24 hr in a dose-dependent manner. Six replicates per group. ***P<0.001, two-tailed t-test. FIG. 10C shows results from a 2-DG uptake assay with ishTDO2 APC-WT and APC-KO MC38 cell lines with and without dox treatment. n=3 biological replicates. **P<0.01, ***P<0.001, two-tailed t-test. FIG. 10D shows measurement of secreted lactate with condition media from ishTDO2 APC-WT and APC-KO MC38 cell lines with and without dox treatment. n=3 biological replicates. *P<0.05, **P<0.01, two-tailed t-test. FIG. 10E shows RT-qPCR analysis of glycolysis pathway genes in ishTDO2 APC-WT and APC-KO MC38 cell lines. n=3 biological replicates. **P<0.01, ***P<0.001, ****P<0.0001, two-tailed t-test. FIG. 10F shows RT-qPCR analysis of glycolysis pathway genes in APC-WT and APC-KO MC38 cell lines expressing shControl or shAhR. n=3 biological replicates. *P<0.05, ***P<0.001, ****P<0.0001, two-tailed t-test. FIG. 10G shows changes in concentration of key glycolysis metabolites in ishTDO2 APC-KO MC38 cell line lysates. The red bars indicate cells without dox treatment and the green bars indicate dox-treated cells. n=5 per biological replicates.

FIGS. 11A-11D show results from studies demonstrating decreased macrophage infiltration by TDO2 knockdown in CRC and BRCA orthotopic models. FIG. 11A shows a representation of IHC staining for F4/80 and CD163 in CRC orthotopic tumor tissues established with ishTDO2 APC-WT and APC-KO MC38 cell lines. Scale bars, 100 μm. FIG. 11B shows a representation of IHC staining for F4/80 and CD163 in CRC orthotopic tumor tissues established with ishTDO2 CT26 cells. Scale bars, 100 μm. FIG. 11C shows a representation of IHC staining for F4/80 and CD163 in CRC orthotopic tumor tissues established with ishTDO2 4T1 cells. Scale bars, 100 μm. FIG. 11D shows that TDO2 mRNA expression is significantly correlated with expression of regulatory T cell markers and MDSC markers in TCGA CRC (COAD+READ, Provisional) patients (n=433). ****P<0.0001.

FIGS. 12A-12G show results from studies demonstrating that the TDO2-AhR-CXCL5 axis regulates macrophage recruitment in APC-mutated CRC tumors. FIG. 12A shows expression of cytokine genes identified in RNA-seq analysis and TDO2 validated by RT-qPCR using ishTDO2 APC-WT and APC-KO MC38 cell lines. n=3 biological replicates. *P<0.05, ***P<0.001, ****P<0.0001. Two-way ANOVA analysis. FIG. 12B shows volume of tumors established with ishTDO2 APC-KO MC38 cell lines expressing Blank, CXCL5, CXCL7, and CSF3. Dox food was applied at day 5 post-orthotopic injection to induce TDO2 knockdown in vivo. n=4 per group. n.s.P>0.05, *P<0.05, ****P<0.001, ****P<0.0001. two-tailed t-test. FIG. 12C shows viSNE analysis of F4/80 and CD206 positive immune cells assessed by CyTOF from CRC orthotopic ishTDO2 APC-WT and APC-KO MC38 tumors and CXCL5-ORF expressing APC-KO MC38 with TDO2 depletion. FIGS. 12D and 12E show quantification of macrophages (F4/80+; FIG. 12D) and M2 macrophages (CD206+; FIG. 12E) in tumors shown in FIG. 12C. CyTOF data analyzed by FlowJo. Data represent mean±s.d., n=3 per group. *P<0.05, **P<0.01. two-tailed t-test. FIG. 12F shows representative images of migrated Raw264.7 cultured with ishTDO2 APC-WT and APC-KO MC38 condition media in transwell assay. Recombinant CXCL5 protein (50 ng) and SX-682 (1 μM) were added to harvested condition media for 36 hr. Scale bar, 100 μm. n=3 biological replicates. Quantification is shown in lower panel. n.s. P>0.05, ***P<0.001, ****P<0.0001. two-tailed t-test. FIG. 12G shows survival curves of C57Bl/6J mice orthotopically implanted with ishTDO2 and ishTDO2/CXCL5-ORF APC-KO MC38 cell lines (5×10⁵ cells). Clondronate liposomes or control encapsome liposomes were given intraperitoneally (100 μl) at day 2 post-orthotopic injection and three times a week. (Encapsome and Clodronate groups, n=6, Dox and Dox+Clodronate groups, n=7, Dox+CXCL5-ORF+Encapsome, n=5, Dox+CXCL5-ORF+Clodronate, n=6). n.s.P>0.05, **P<0.01, ***P<0.001. two-tailed t-test.

FIGS. 13A-13I show results from studies demonstrating that CXCL5 is regulated by the TDO2-AhR pathway and is responsible for macrophage recruitment in APC-mutant CRC. FIG. 13A shows a cytokine array of conditioned media from ishTDO2 APC-KO MC38 cells with and without dox treatment. The red boxes (labeled as 1-6) indicate cytokines that significantly decreased after TDO2 knockdown. FIG. 13B shows a representation of migrated BMDM cultured in condition media from APC-WT and APC-KO MC38 cell lines expressing shControl or shTDO2. n=3 biological replicates. Scale bars, 50μm. FIG. 13C shows a ChIP-seq in APC-KO MC38 cells demonstrating binding peaks for AhR on the promoters of CXCL5 gene. FIG. 13D shows RT-qPCR analysis for expression of cytokines after AhR knockdown in APC-KO MC38 cell lines. n=3 biological replicates. FIG. 13E shows RT-qPCR analysis of M2 macrophage signature genes in Raw264.7 cells incubated with conditioned media from ishTDO2 APC-WT and APC-KO MC38 cells for 30 h. n=3 biological replicates. FIG. 13F shows RT-qPCR analysis of M2 macrophage signature genes Arg1 and YM1 and M1 macrophage signature gene iNOS in BMDM treated with CSF1 (50 ng), CXCL5 (50 ng), and Kyn (1 mM) for 24 hr. n=3 biological replicates. n.s.P>0.05, ***P<0.001, ****P<0.0001. two-tailed t-test. FIG. 13G shows a representation of IHC staining for F4/80 and CD163 in CRC orthotopic tumor tissues established with ishTDO2 and ishTDO2/CXCL5-ORF APC-KO MC38 cell lines. Scale bars, 100 μm. FIG. 13H shows survival curves of C57Bl/6J mice orthotopically implanted with ishTDO2 APC-KO MC38 cell lines expressing shCXCL5 (5×10⁵ cells). (isTDO2 and isTDO2/shCXCL5 #2 groups, n=7, isTDO2/shCXCL5 #3, n=8). ****P<0.0001. two-tailed t-test. FIG. 13I shows survival curves of C57Bl/6J mice orthotopically implanted with ishTDO2 APC-KO MC38 cell lines (5×10⁵ cells). (shControl and shCXCL5 #2 groups, n=7, shCXCL5 #3, n=8). ****P<0.0001. two-tailed t-test.

FIGS. 14A-14G show results from studies demonstrating that CXCL5 expression is correlated with macrophage population and tryptophan metabolism in CRC and BRCA. FIGS. 14A and 14B show GSEA correlation of macrophage populations with CXCL5-High tumors in TCGA CRC and BRCA database. NES and nominal P value are shown. FIGS. 14C and 14D show a list of immune populations positively correlated with CXCL5 expression in TCGA CRC (n=254) and BRCA (n=730) tumors. The highlighted bar indicates macrophages. NES values are shown. FIG. 14E shows GSEA correlation of tryptophan metabolism and xenobiotic metabolism with CXCL5-High TCGA CRC tumors. Normalized enrichment score (NES) and nominal P value are shown. FIG. 14F shows representative images of clustered TCGA CRC patient samples based on CXCL5 expression and expression of correlative top six tryptophan metabolism-associated genes (TDO2, KYNU, KMO, IDO1, IL4I1, and AOX1) in ranked order (CXCL5 high, n=127, CXCL5 low, n=127). FIG. 14G shows GSEA correlation of tryptophan metabolism and xenobiotic metabolism with CXCL5-High TCGA BRCA tumors. Normalized enrichment score (NES) and nominal P value are shown.

FIGS. 15A-15K show results from studies demonstrating that Gas6 secreted by macrophages binds to Axl on tumor cells and promotes tumor growth. FIG. 15A shows a mouse RTK phospho-array of lysates from ishTDO2 APC-WT and APC-KO MC38 cell lines before and after dox treatment. The red box indicates Axl. FIG. 15B shows immunoblots for Axl and phospho-Axl in APC-WT and APC-KO MC38 cells after R428 treatment (0.2 μM, 24 h). FIG. 15C shows immunoblots for Axl and phospho-Axl in APC-WT and APC-KO MC38 cells after supplementing recombinant mouse Gas6 protein (rmGAS6) for 24 h. FIG. 15D shows a representation of colony formation assay of LS180 and HT-29 cell lines treated with Gas6 (25 ng) only and with R428 (1 μM) for 36 hr. n=3 biological replicates. FIG. 15E shows results from a cell viability assay of APC-WT and APC-KO MC38 cells treated with 0.5 μM R428 for 48 h in a dose-dependent manner. Six replicates per group. two-tailed t-test. *P<0.05. FIG. 15F shows immunofluorescence staining of phosphorylated Axl (RFP) in CRC orthotopic tumors established by injecting APC-WT and APC-KO MC38 cells into C57BL/6J mice. Epithelial cells are stained with EpCam and indicated by GFP. X63 magnification. FIG. 15G shows RT-qPCR analysis of Gas6 expression in BMDM cultured in condition media from ishTDO2 APC-WT and APC-KO MC38 cell lines. n=3 biological replicates. n.s.P>0.05, ****P<0.0001. two-tailed t-test. FIG. 15H shows RT-qPCR analysis of Gas6 expression in BMDM treated with CSF1 (50 ng), Kyn (1 mM), CXCL5 (50 ng) and Gas6 (25 ng) for 24 hr. n=3 biological replicates. ****P<0.0001. two-tailed t-test. FIG. 15I shows RT-qPCR shows GAS6 shRNA knockdown efficiency in Raw264.7. n=3 biological replicates. FIG. 15J shows representative in vivo bioluminescence-based images of BalB/CJ mice at day 20 post-orthotopic co-injection of CT26 cells (1×10⁵ cells) and macrophage cell line Raw264.7 (1×10⁵ cells) with shControl or shGAS6. Total flux measurement of at the end point. **P<0.01. two-tailed t-test. FIG. 15K shows representative in vivo bioluminescence-based images of BalB/CJ mice at day 20 post-orthotopic co-injection of CT26 cells (1×10⁵ cells) and macrophage cell line Raw264.7 (1×10⁵ cells) pre-treated with 100 ng of CXCL5 for 48 h before injection. Total flux measurement of tumors was done at the end point. **P<0.01. two-tailed t-test.

FIG. 16 shows a schematic of a working model for activation of the TDO2-Kyn-AhR-CXCL5 axis and recruitment of TAMs in APC-deficient CRC cells.

### DETAILED DESCRIPTION

Inactivation of the tumor suppressor adenomatous polyposis coli (APC) is the critical initiating event for development of the majority (˜90%) of sporadic colorectal cancers (CRC). APC loss also occurs frequently across many other cancer types. The APC signaling cascade has remained an elusive target for cancer therapy. Using the concept of synthetic essentiality, Tryptophan 2,3-dioxygenase 2 (TDO2) is identified herein as a synthetic essential (SE) gene for APC-deficient cancers. Mechanistically, activation of TDO2-Kyn-AhR axis drives glycolysis and promotes cell proliferation and growth in APC-deficient CRC cells as well as upregulates CXCL5 which recruits tumor associated macrophages (TAMs). These TAMs promote an immunosuppressive tumor microenvironment (TME) as well as support cancer cell survival via their secretion of GAS6 which activates AXL in cancer cells. Thus, APC-deficiency creates a TDO2-driven circuit that establishes a symbiotic relationship between cancer cells and infiltrating TAMs in the CRC TME. Disclosed herein, in some embodiments, are methods and compositions for targeting TDO2 and downstream effectors (e.g., cytokines) for treatment of APC-deficient tumors.

### I. TDO2 NUCLEIC ACIDS AND POLYPEPTIDES

Tryptophan 2,3-dioxygenase 2 (TDO2) is known in the art and exemplified by the following DNA, mRNA, and protein sequences described herein. In some embodiments, TDO2 is an enzyme having Enzyme Commission (EC) classification number 1.13.11.11.

For example, the TDO2 gene may be exemplified by Homo sapiens tryptophan 2,3-dioxygenase (NCBI reference sequence: NC_000004.12)

In another example, the TDO2 mRNA is exemplified by Homo sapiens tryptophan 2,3-dioxygenase (TDO2), mRNA (NCBI reference sequence: NM_005651.4).

In another example, the TDO2 protein is exemplified by Homo sapiens tryptophan 2,3-dioxygenase (NCBI reference sequence: NP_005642.1):

(SEQ ID NO: 1)
MSGCPFLGNNFGYTFKKLPVEGSEEDKSQTGVNRASKGGLIYGNYLHLEK
VLNAQELQSETKGNKIHDEHLFIITHQAYELWFKQILWELDSVREIFQNG
HVRDERNMLKVVSRMHRVSVILKLLVQQFSILETMTALDFNDFREYLSPA
SGFQSLQFRLLENKIGVLQNMRVPYNRRHYRDNFKGEENELLLKSEQEKT
LLELVEAWLERTPGLEPHGFNFWGKLEKNITRGLEEEFIRIQAKEESEEK
EEQVAEFQKQKEVLLSLFDEKRHEHLLSKGERRLSYRALQGALMIYFYRE
EPRFQVPFQLLTSLMDIDSLMTKWRYNHVCMVHRMLGSKAGTGGSSGYHY
LRSTVSDRYKVFVDLFNLSTYLIPRHWIPKMNPTIHKFLYTAEYCDSSYF
SSDESD.

In some embodiments, the TDO2 polypeptide or nucleic acid comprises a human TDO2 polypeptide or human TDO2 nucleic acid. In some embodiments, the TDO2 polypeptide or TDO2 nucleic acid is non-human. In some embodiments, the TDO2 polypeptide or TDO2 nucleic acid is from mouse, horse, dog, rabbit, or goat.

### II. INHIBITORS

Aspects of the present disclosure include inhibitors of TDO2 and/or inhibitors of a cytokine activated by TDO2 activity.

In some embodiments, methods and compositions of the present disclosure comprise an inhibitor of TDO2. An inhibitor of TDO2 may describe any molecule capable of inhibiting expression and/or activity of TDO2. In some embodiments, a TDO2 inhibitor is a small molecule inhibitor, which may serve to inhibit the enzymatic activity of TDO2. Examples of small molecule TDO2 inhibitors include PF06845102/EOS200809, 680C91, LM10, HTI-1090, DN1406131, RG70099, EPL-1410, CB548, CMG017, and derivatives thereof. In some embodiments, a TDO2 inhibitor is a dual TDO2/IDO1 inhibitor. In some embodiments, a TDO2 inhibitor is a dual TDO2/IDO2 inhibitor. In some embodiments, a TDO2 inhibitor is a selective TDO2 inhibitor. In some embodiments, a TDO2 inhibitor is a nucleic acid capable of inhibiting TDO2 expression in a cell. In some embodiments, a TDO2 inhibitor is a small interfering RNA (siRNA), small hairpin RNA (shRNA), antisense oligonucleotide, morpholino, or similar nucleic acid designed to target and inhibit translation of TDO2 mRNA. In some embodiments, the TDO2 inhibitor is an antisense oligonucleotide complementary to all or a portion of a TDO2 messenger RNA encoding for a TDO2 protein (SEQ ID NO: 1). In some embodiments, a TDO2 inhibitor is an antibody, antibody-like molecule, or other TDO2 binding protein.

In some embodiments, methods and compositions of the present disclosure comprise an inhibitor of a cytokine activated by TDO2 activity. A cytokine activated by TDO2 activity may describe a cytokine whose expression is increased as a result of the enzymatic activity of TDO2. Cytokines activated by TDO2 activity include, but are not limited to, CXCL5, CXCL7, CSF3, CXCR2, CXCL2, CXCL10, CCL2, and CXCL1. In some embodiments, the disclosed methods comprise providing an inhibitor of CXCL5. In some embodiments, an inhibitor of a cytokine activated by TDO2 activity is a nucleic acid capable of inhibiting expression of the cytokine in a cell. In some embodiments, such an inhibitor is a siRNA, shRNA, antisense oligonucleotide, or similar nucleic acid designed to target and inhibit translation of the cytokine mRNA. In some embodiments, the TDO inhibitor is an antisense oligonucleotide complementary to all or a portion of a messenger RNA encoding for a cytokine activated by TDO2 activity (e.g., CXCL5, CXCL7, CSF3, CXCR2, CXCL2, CXCL10, CCL2, or CXCL1). In some embodiments, an inhibitor of a cytokine activated by TDO2 activity is an antibody, antibody-like molecule, or other binding protein capable of specifically binding to a cytokine activated by TDO2 activity. In some embodiments, the disclosed methods comprise providing an antibody capable of specifically binding to CXCL5, CXCL1, or CXCL2. In some embodiments, the disclosed methods comprise providing an antibody capable of specifically binding to CXCL5.

A. Inhibitory Oligonucleotides

In some aspects, the disclosure relates to inhibitory oligonucleotides that inhibit the gene expression of TDO2. Examples of an inhibitory oligonucleotides include but are not limited to siRNA (small interfering RNA), short hairpin RNA (shRNA), double-stranded RNA, an antisense oligonucleotide, and a ribozyme. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of TDO2 by at least 10%, 20%, 30%, 40%, 50%, 60%, 70%, 75%, 80%, 90%, 95%, 99%, or 100% or any range or value in between the foregoing.

In further embodiments, disclosed are synthetic oligonucleotides that are TDO2 inhibitors. An inhibitor may be between 17 to 25 nucleotides in length and comprises a 5′ to 3′ sequence that is at least 90% complementary to the 5′ to 3′ sequence of a mature TDO2 mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature TDO2 mRNA, particularly a mature, naturally occurring mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

### III. THERAPEUTIC METHODS

The compositions of the disclosure may be used for in vivo, in vitro, or ex vivo administration. The route of administration of the composition may be, for example, intracutaneous, subcutaneous, intravenous, local, topical, and intraperitoneal administrations.

In some embodiments, the disclosed methods are directed to methods for treating cancer. The cancer may be a solid tumor, metastatic cancer, or non-metastatic cancer. In certain embodiments, the cancer may originate in the bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus. In some embodiments, the cancer originates in the colon. In some embodiments, the cancer originates in the rectum.

The cancer may specifically be of one or more of the following histological types, though it is not limited to these: undifferentiated carcinoma, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophogogastric, glioblastoma, adrencorical, vueal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; thymoma; thecoma; androblastoma; sertoli cell carcinoma; malignant melanoma; amelanotic melanoma; superficial spreading melanoma; epithelioid cell melanoma; sarcoma; mesenchymal (e.g., fibrosarcoma; fibrous histiocytoma; myxosarcoma; liposarcoma; leiomyosarcoma; rhabdomyosarcoma; embryonal rhabdomyosarcoma; alveolar rhabdomyosarcoma; stromal sarcoma; mullerian mixed tumor; nephroblastoma; hepatoblastoma; carcinosarcoma; dysgerminoma; embryonal carcinoma; choriocarcinoma; mesonephroma; hemangiosarcoma; Kaposi's sarcoma; lymphangiosarcoma; osteosarcoma; juxtacortical osteosarcoma; chondrosarcoma; mesenchymal chondrosarcoma; giant cell tumor of bone; Ewing's sarcoma; ameloblastic odontosarcoma; ameloblastic fibrosarcoma; chordoma; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; neurofibrosarcoma; paragranuloma); or hematopoietic (e.g., multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia).

In some embodiments, methods of the present disclosure comprise treating cancer, where the cancer is colorectal cancer, breast cancer, prostate cancer, lung cancer, head and neck squamous cell carcinoma, or sarcoma. In some embodiments, the cancer is colorectal cancer (CRC). In some embodiments, the cancer is a cancer having constitutively active WNT signaling. In some embodiments, the cancer is an APC-deficient cancer. In some embodiments, the cancer is an APC-deficient CRC.

As used herein, an “APC-deficient cancer” describes a cancer which has reduced expression and/or activity of an adenomatous polyposis coli (APC) gene or protein relative to a normal or non-cancerous cell. In one example, an APC-deficient cancer is a cancer having a mutation in the APC gene or a regulatory region of the APC gene, where the mutation prevents expression of APC. In another example, an APC-deficient cancer is a cancer having a mutation in the APC gene, where the mutation prevents the APC protein from functioning normally (e.g., causes misfolding of the protein). In another example, an APC-deficient cancer is a cancer having epigenetic repression of APC gene expression.

Certain aspects of the disclosure are directed to therapeutic methods for treating a subject with an APC-deficient cancer. In some embodiments, the disclosed methods comprise detecting an APC deficiency (e.g., mutation, epigenetic repression, etc.) in cancer cells from a subject prior to treatment, for example via DNA sequencing, polymerase chain reaction, or other suitable molecular technique. In some embodiments, the methods comprise treating a subject known to have a cancer with an APC deficiency. In some embodiments, the methods comprise treating a subject presumed to have a cancer with an APC deficiency. For example, in some embodiments, disclosed is a method of treating a subject with colorectal cancer, where the colorectal cancer is presumed to have an APC deficiency. In some embodiments, the disclosed methods do not comprise a step of detecting an APC deficiency in cancer cells from a subject.

In some embodiments, the methods comprise detecting an APC deficiency in healthy (e.g., non-cancerous) cells from a subject. In some embodiments, methods comprise treating a subject known to have an APC deficiency. For example, methods may comprise diagnosing a subject with familial adenomatous polyposis (FAP) and/or treating a subject known to have FAP. In some embodiments, the disclosed methods do not comprise a step of detecting an APC deficiency in healthy cells from a subject.

### IV. IMMUNOTHERAPY

In some embodiments, the disclosed methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Certain immunotherapies are known in the art, and some are described below.

1. Activation of Co-Stimulatory Molecules

In some embodiments, the immunotherapy comprises an agonist of a co-stimulatory molecule. In some embodiments, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include agonistic antibodies, polypeptides, compounds, and nucleic acids.

A. Checkpoint Inhibitors and Combination Treatment

Embodiments of the disclosure may include administration of immune checkpoint inhibitors, which are further described below.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

C. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta). In some embodiments, the CAR-T therapy targets CD19.

D. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

E. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### V. SAMPLE PREPARATION

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VI. ADMINISTRATION OF THERAPEUTIC COMPOSITIONS

The therapy provided herein may comprise administration of a combination of therapeutic agents, such as a first cancer therapy (e.g., a TDO2 inhibitor) and a second cancer therapy (e.g., an additional cancer therapy such as chemotherapy, radiation therapy, or immunotherapy). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 pg/kg, mg/kg, pg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of pg/kg or mg/kg of body weight can be converted and expressed in comparable concentration units of pg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. KITS

Certain aspects of the present disclosure also concern kits containing compositions of the disclosure or compositions to implement methods disclosed herein. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers. In some embodiments, a control includes a nucleic acid that contains at least one CpG or is capable of identifying a CpG methylation site.

It is contemplated that any method or composition described herein can be implemented with respect to any other method or composition described herein and that different embodiments may be combined. The claims originally filed are contemplated to cover claims that are multiply dependent on any filed claim or combination of filed claims.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### VIII. CANCER THERAPY

In some embodiments, the disclosed methods comprise administering a cancer therapy to the patient. The cancer therapy may be chosen based on the expression level measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. In some embodiments, the cancer therapy comprises treatment with an IDO2 inhibitor. In some embodiments, the cancer therapy comprises treatment with an inhibitor of a cytokine activated by IDO2 activity. Any of these cancer therapies may also be excluded. Combinations of these therapies may also be administered. In some embodiments, the gene or miRNA expression measurement and analysis may indicate that one or more cancer therapies would be likely to be effective or ineffective.

### IX. CELLULAR THERAPIES

A. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., p-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### X. DETECTING A GENETIC SIGNATURE

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

C. DNA Sequencing

In some embodiments, DNA may be analyzed by sequencing. The DNA may be from an individual (e.g., from the germline of an individual). The DNA may be from a cancer cell. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar. Sequencing may be used to determine a mutation in a gene. Sequencing may also be used to identify a methylation status of a gene (e.g., bisulfite sequencing or other methylation-sensitive sequencing).

D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of pg of enzyme to pg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

F. Detection Kits and Systems

One can recognize that based on the methods described herein, detection reagents, kits, and/or systems can be utilized to detect the SNP and/or the CNV related to the genetic signature for diagnosing an individual (the detection either individually or in combination). The reagents can be combined into at least one of the established formats for kits and/or systems as known in the art. As used herein, the terms “kits” and “systems” refer to embodiments such as combinations of at least one SNP detection reagent, for example at least one selective oligonucleotide probe, and at least one CNV detection reagent, for example at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

### XI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification of TDO2 as Downstream Effector for APC Deficiency in Cancer

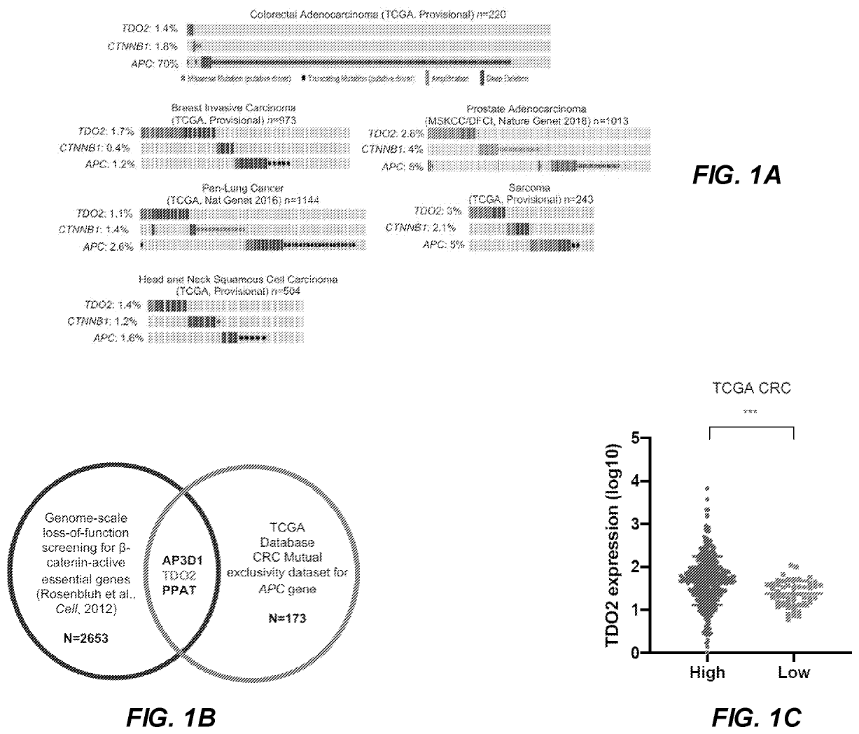

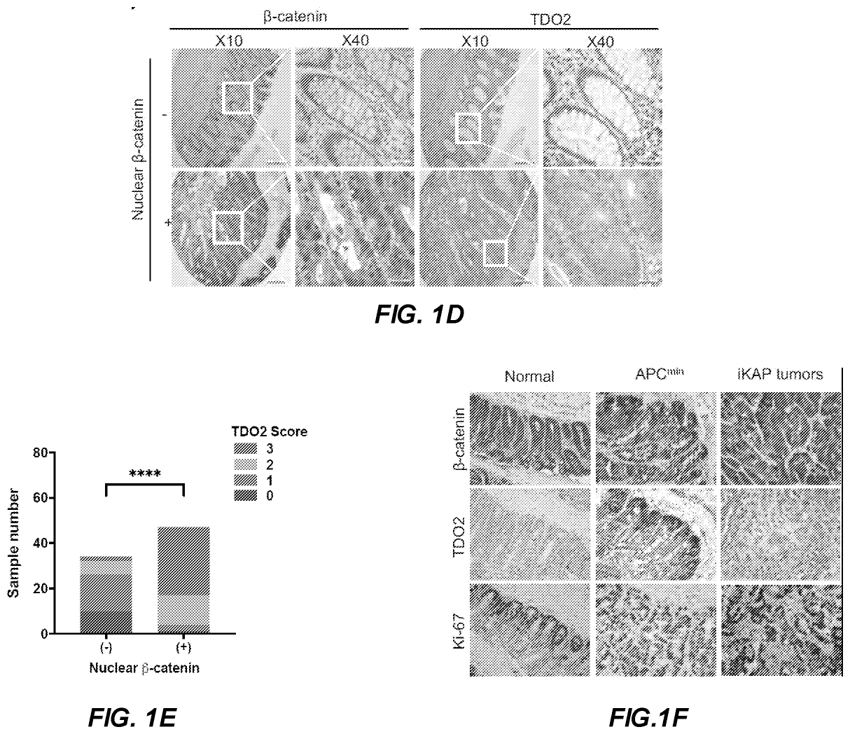

To identify downstream effectors of APC deficient tumors, specifically synthetic essential (SE) genes, genes were identified that showed mutually exclusive mutation/deletion patterns with APC/beta-catenin in The Cancer Genome Atlas (TCGA) database. To overcome the limitation that only a small fraction of CRC cases are intact for APC, a pan-cancer analysis was conducted which showed consistent retention of tryptophan 2,3-dioxygenase 2 (TDO2) in APC deleted/mutated cancers including CRC, breast cancer, prostate cancer, lung cancer, head and neck squamous cell carcinoma, and sarcoma (FIG. 1A). Recognizing the limited sample size and low frequency of these genomic events, these genomic results were then intersected with hits from genome wide loss-of-function screens designed to identify genes that are consistently retained in cancer cells bearing APC loss-of-function mutations (Rosenbluh et al. Cell. 2012 Dec. 21; 151(7):1457-73; incorporated herein by reference in its entirety). This intersection yielded 3 candidate SE genes for APC deficient tumors: TDO2, adaptor related protein complex 3 subunit delta 1 (AP3D1) and phosphoribosyl pyrophosphate amidotransferase (PPAT) (FIG. 1B).

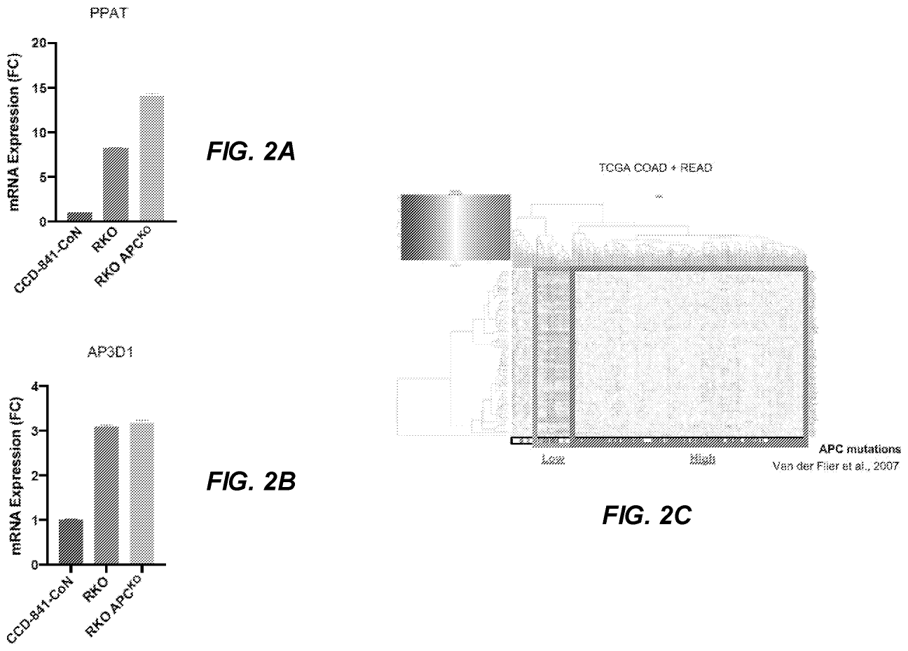

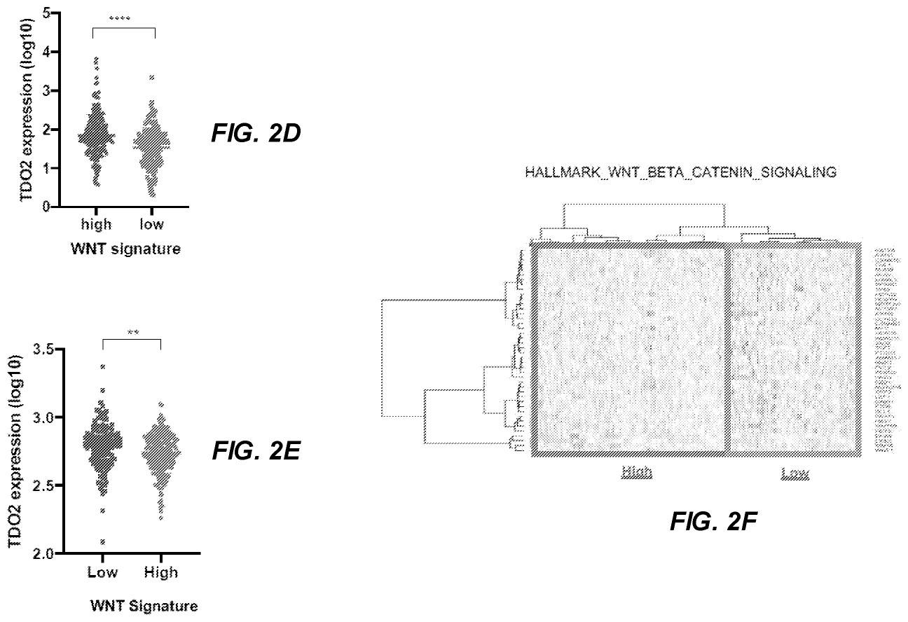

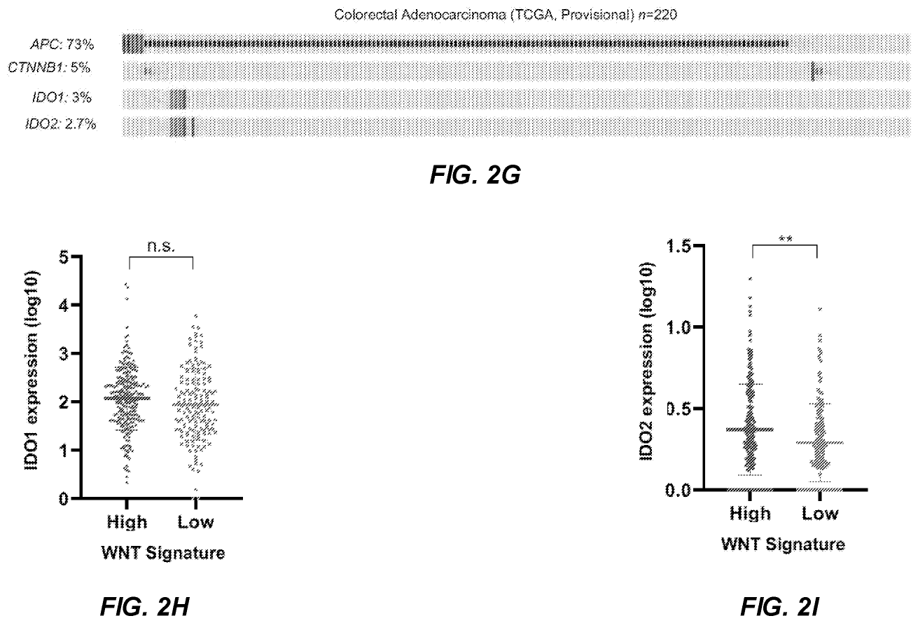

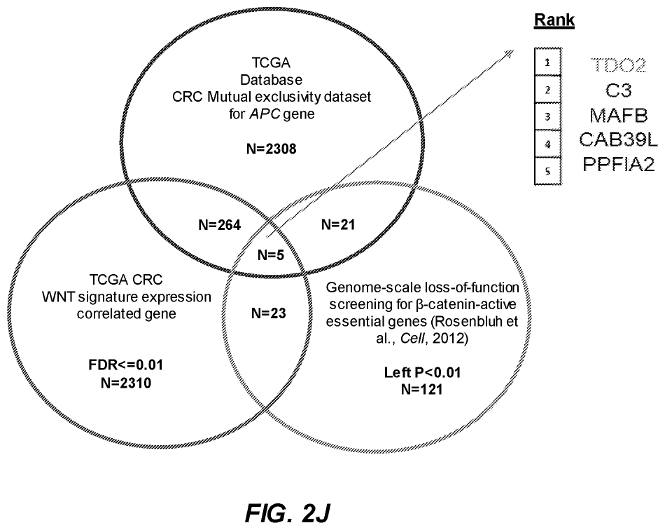

Isogenic APC-WT and APC-KO RKO cell lines were generated and APC deletion was found to increase gene expression of TDO2 and PPAT (FIGS. 2A and 2B), suggesting a potential genetic link between APC and TDO2 or PPAT. In addition, unbiased transcriptomic analyses assessed possible correlations of WNT pathway activation signature (Van der Flier et al., 2007) and TDO2 expression in CRC datasets, revealing that TDO2 gene expression correlated positively with WNT pathway activation (FIG. 1C; FIGS. 2C-2F). Correspondingly, tumor microarray analysis of human CRC samples showed coincident nuclear beta-catenin (surrogate of an activated WNT pathway) and TDO2 expression (FIGS. 1D and 1E). Finally, in the mouse, TDO2 expression was undetectable in the normal colon epithelial cells yet dramatically increased in polyps of APC mutant mice (APCmin) and in tumors of iKAP mice (inducible Krasmut with APCmut/TP53mut) (Boutin et al., 2017) (FIG. 1F). In contrast, the other kynurenine pathway (KP) enzymes, IDO1 and IDO2, did not exhibit mutual exclusive patterns with APC and beta-catenin (CTNNB1) mutations in TCGA CRC nor show correlations with WNT pathway activation (FIGS. 2G-2I). Collectively, these observations indicated that TDO2 may be a downstream effector of APC deficiency with potential biological and clinical relevance in CRC and other cancer types.

A similar analysis was performed using the same datasets with statistical cutoffs for each dataset. TDO2 was the highest ranked gene identified by the triangulation. Specifically, triangulation identified 5 genes (TDO2, C3, MAFB, CAB39L, PPFIA2) with cutoffs of p<0.01 and FDR<=0.01 (FIG. 2J).

### Example 2—APC Deficiency Upregulates TDO2 Expression Via TCF4

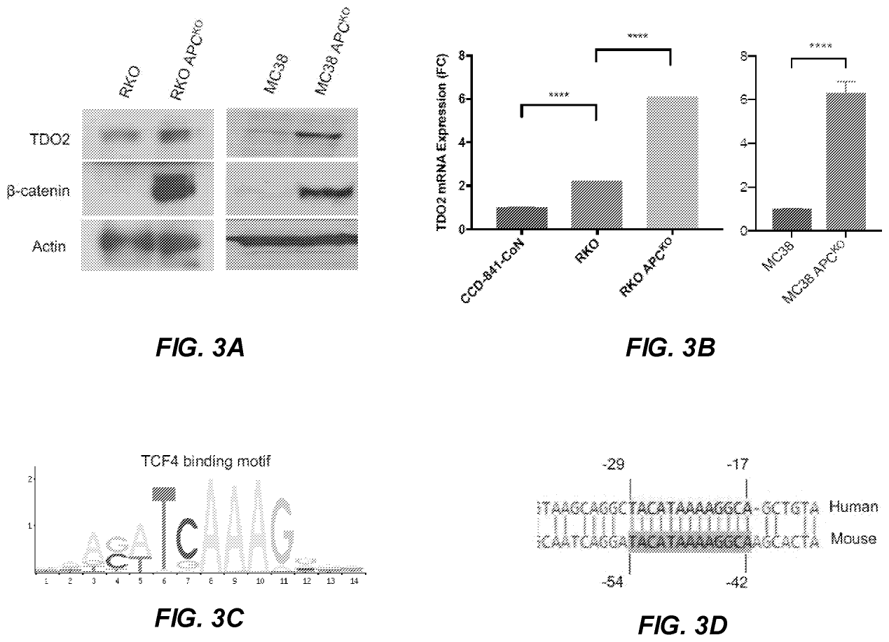

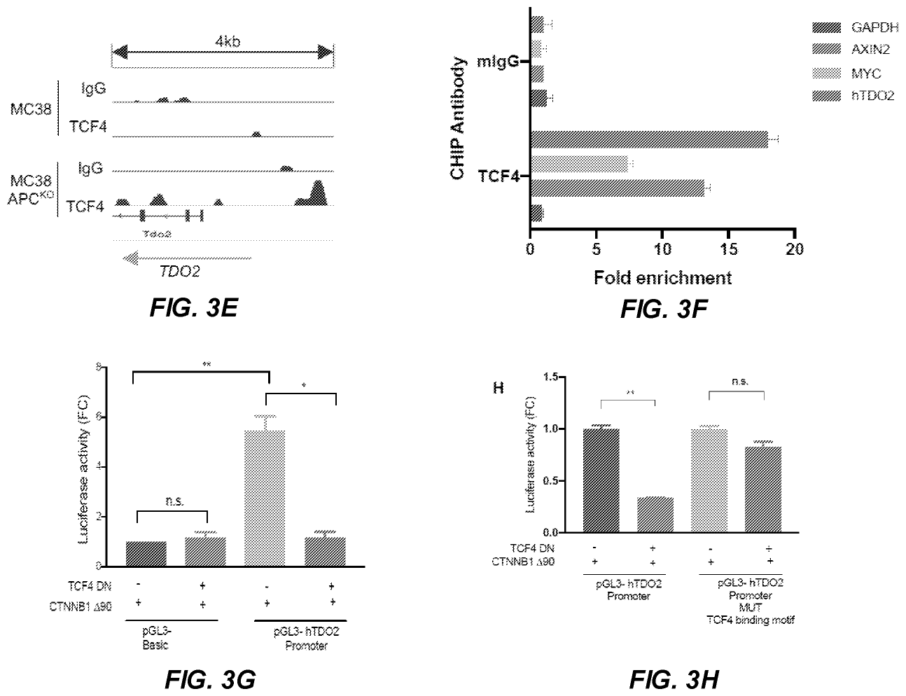

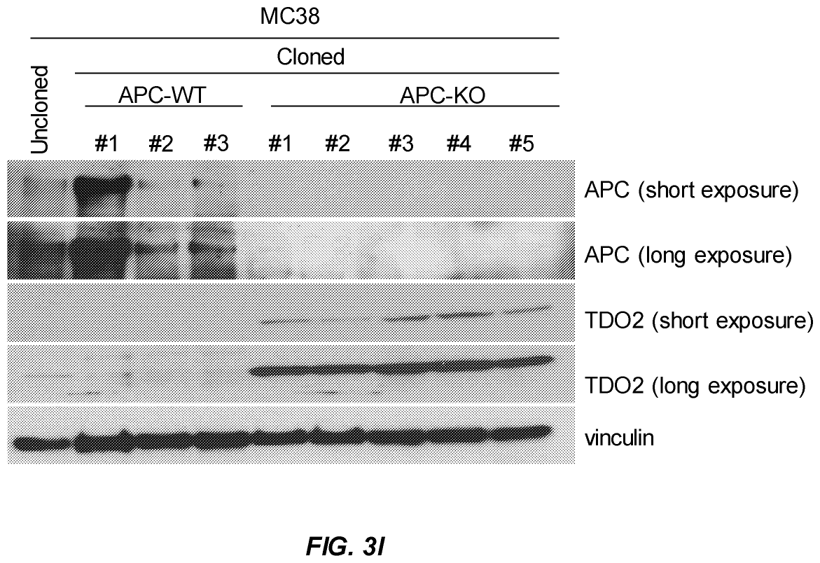

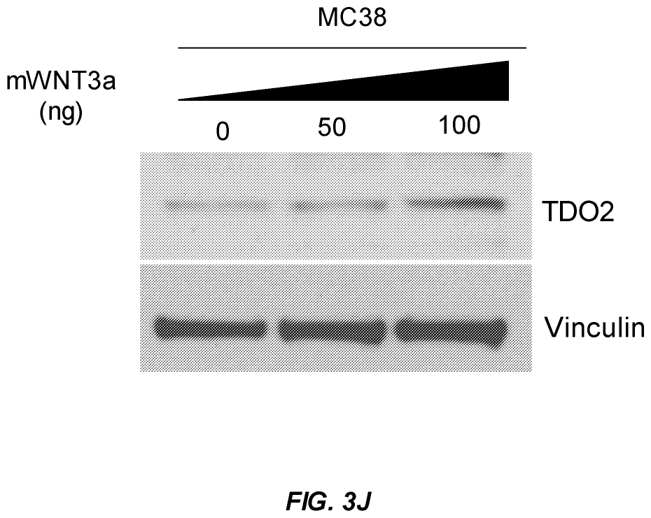

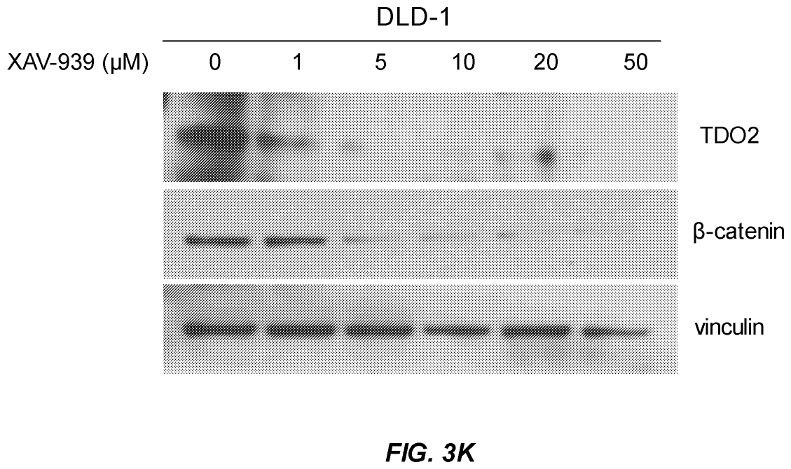

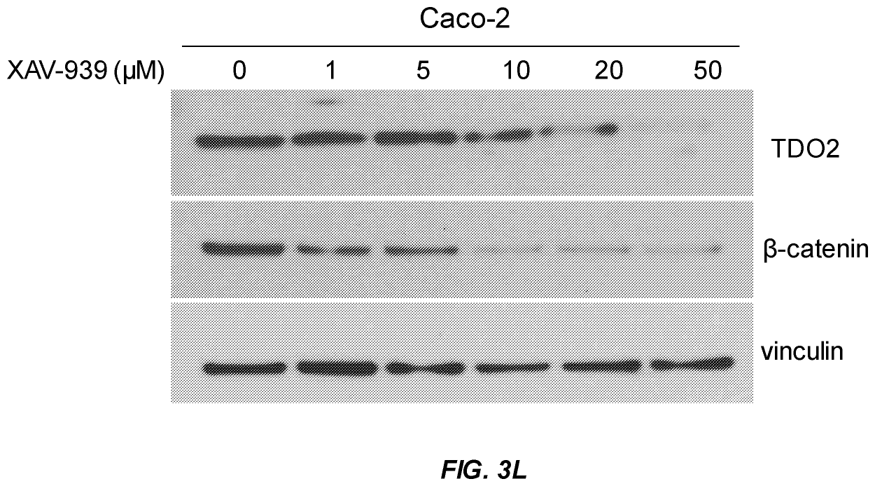

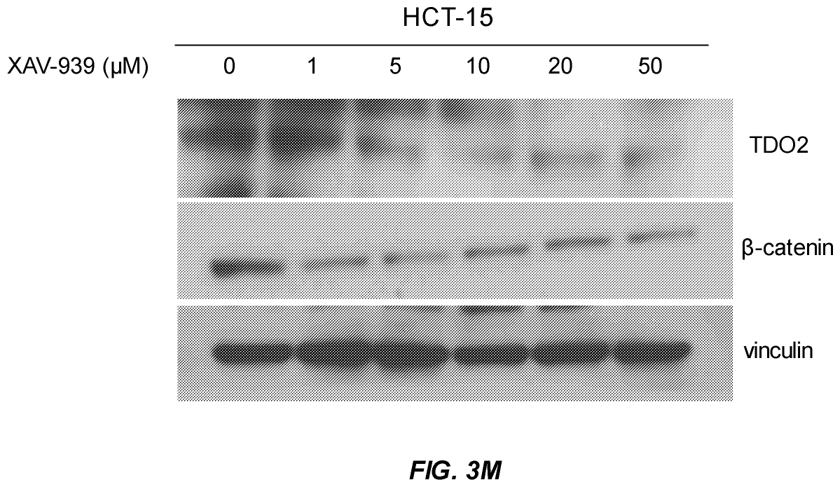

APC deletion increased TDO2 mRNA and protein levels (FIGS. 3A and 3B), prompting examination of the promoter regions of the human and mouse TDO2 genes for binding elements of WNT pathway-related transcription factors. Using transcription factor binding profile databases JASPAR and ECR Browser, the upstream regions of TDO2 gene were found to harbor binding sites for TCF4/TCF7L2, a known transcription factor mediating WNT pathway (FIG. 3C). One of the motif sequences is located close to the TSS and conserved in human and mouse (FIG. 3D).

ChIP-seq analysis using an anti-TCF4 antibody showed specific binding of TCF4 to the TDO2 promoter region in APC-KO, but not APC-WT, MC38 cells (FIG. 3E). Additional CHIP-PCR analysis in the APC null DLD1 cell line also documented TCF4 binding directly to the TDO2 promoter as well as well-known WNT pathway targets AXIN2 and MYC but not GAPDH which serves as a negative control (FIG. 3F). Accordingly, a luciferase reporter assay employing a construct driven by human TDO2 promoter showed that transduction of constitutively active beta-catenin (CTNNB1 Δ90, which mimics the condition of WNT pathway activation) results in increased reporter gene transcription. In contrast, reporter gene expression decreased with expression of dominant-negative form of TCF4 or with mutation of TCF4 binding motif (FIGS. 3G and 3H). To eliminate clonal variation effect, multiple APC-KO MC38 clones were generated to confirm the status of TDO2 expression (FIG. 3I). Each of the APC-KO clones showed higher TDO2 expression relative to uncloned control cells and APC-WT clones. In addition, treatment of APC-WT MC38 cells with recombinant mouse WNT3a protein increased the TDO2 expression (FIG. 3J), suggesting that the endogenous activation of WNT signaling can increase the expression of TDO2.

Caco-2, DLD-1, and HCT-15 cell lines were treated with the WNT inhibitor XAV-939 in a dose-dependent manner and TDO2 expression was decreased by inhibited WNT activity indicated by the reduced level of β-catenin (FIGS. 3K-3M). Thus, APC loss induced activation of the WNT-betacatenin-TCF4 pathway resulting in TCF4-mediated upregulation of TDO2 gene transcription.

### Example 3—TDO2 Depletion Specifically Impairs Growth and Survival of APC/WNT Mutated-CRC Cells

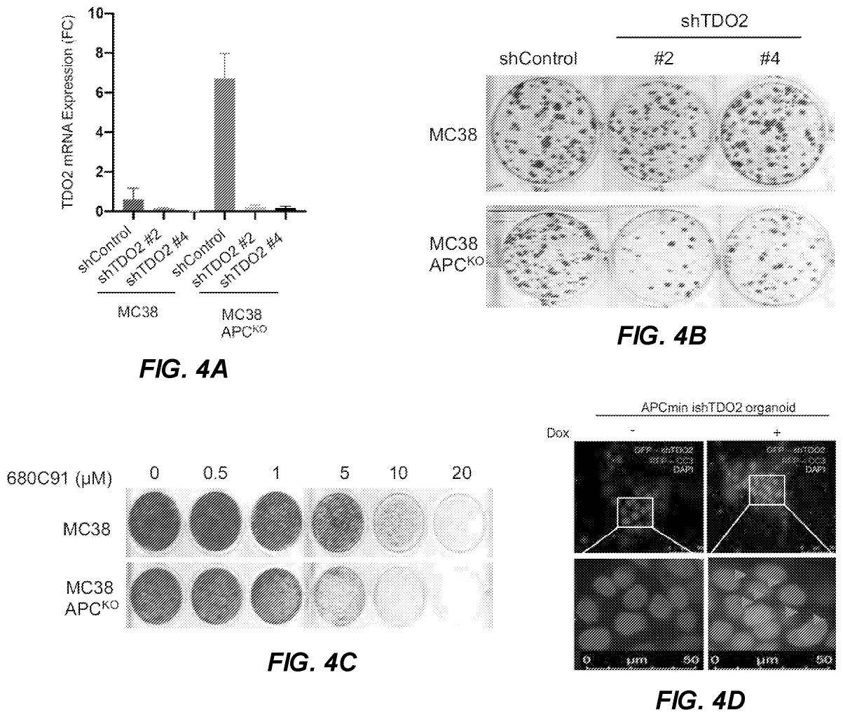

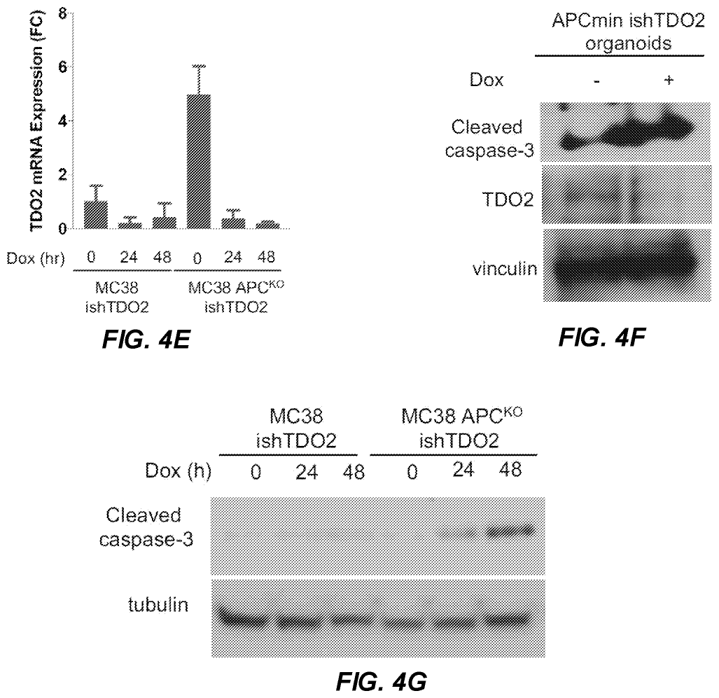

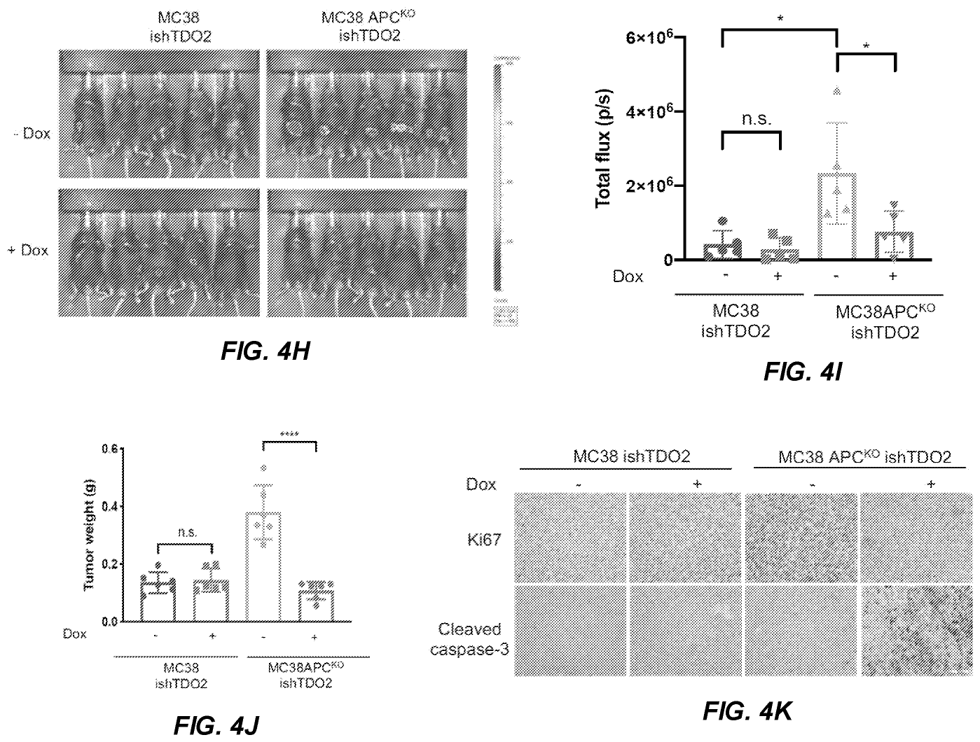

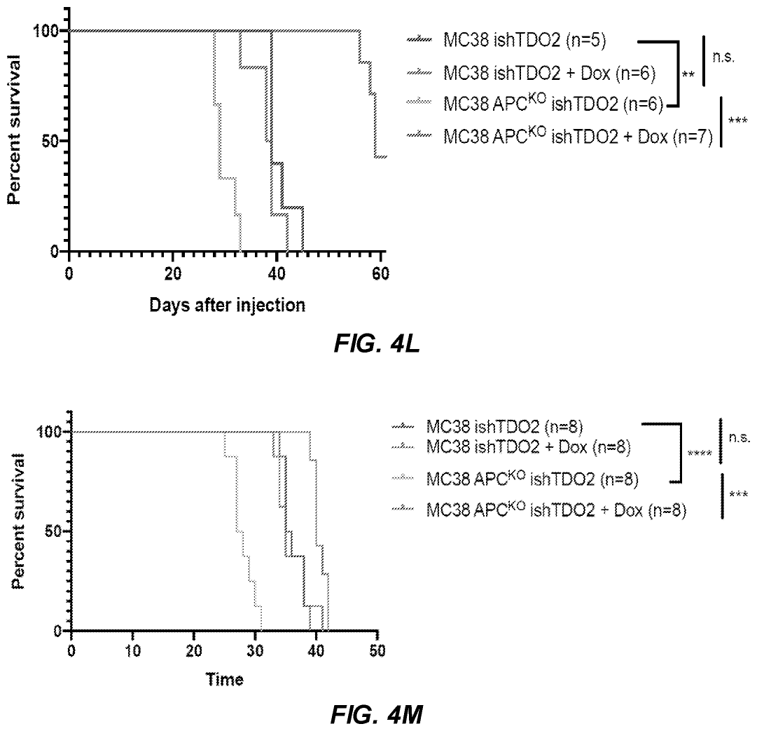

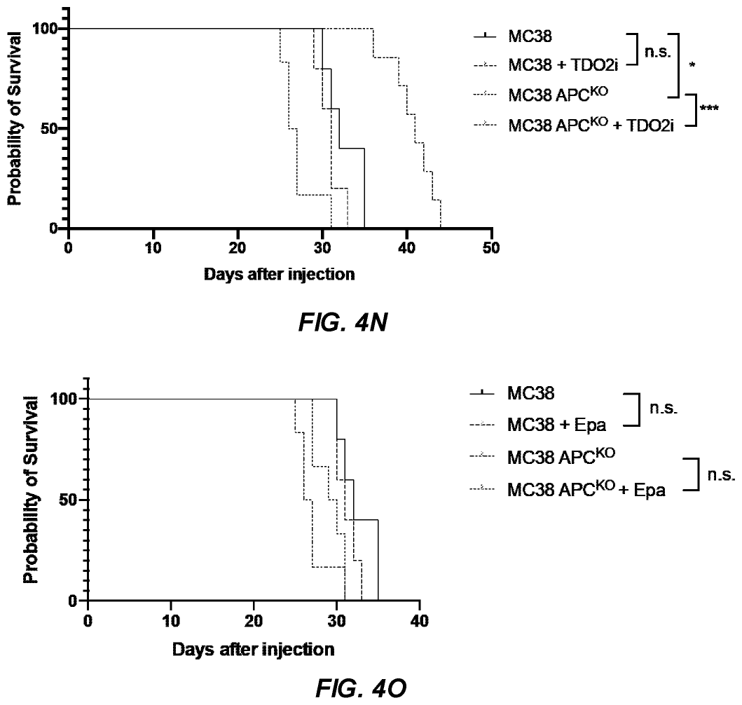

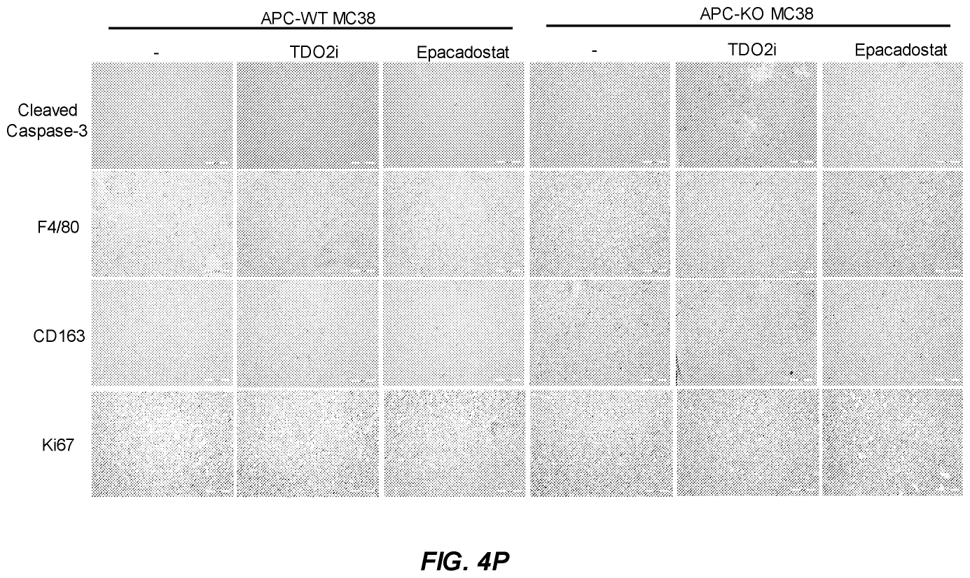

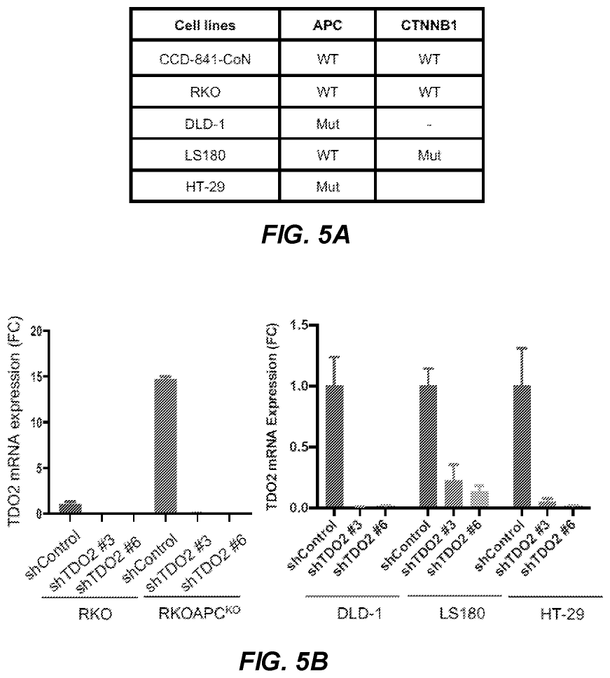

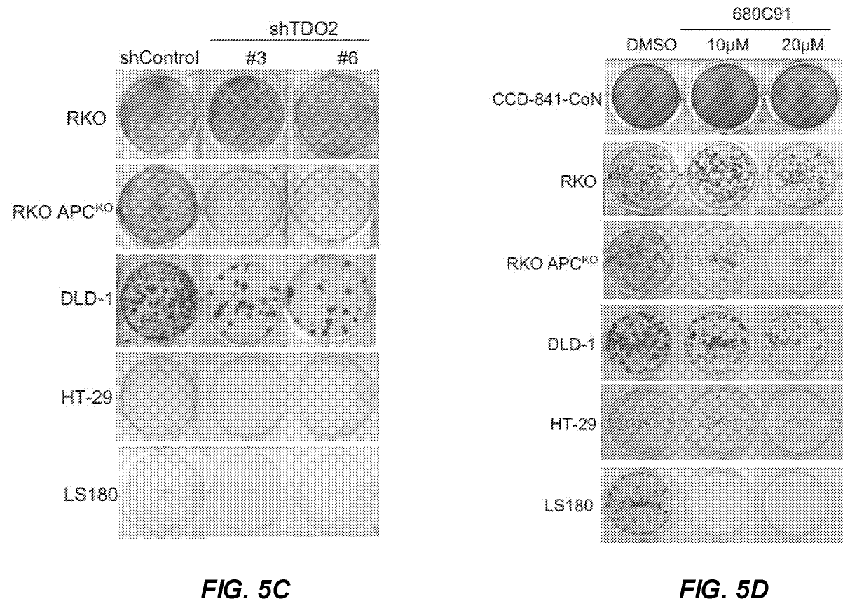

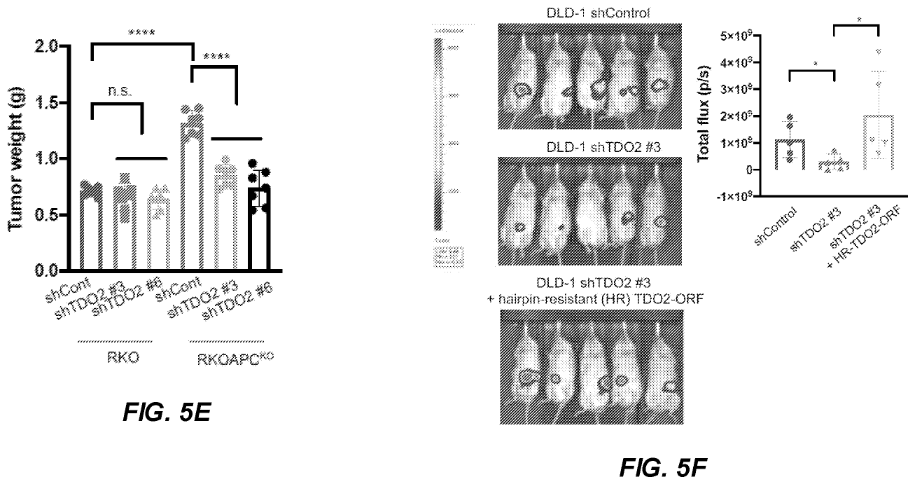

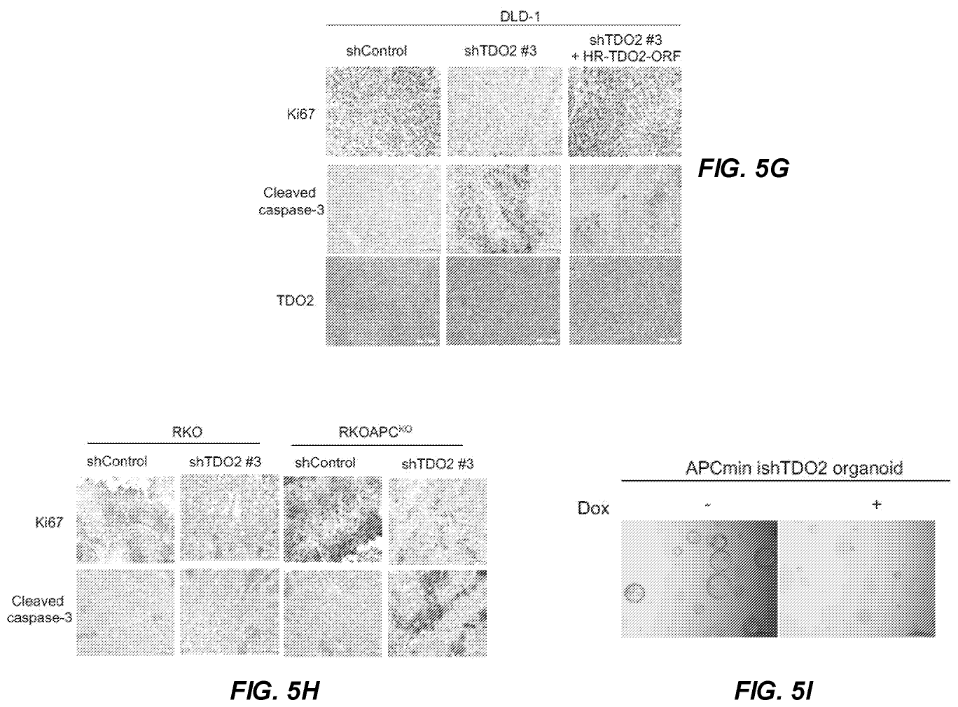

To test whether TDO2 is specifically essential for APC-deficient cells, human APC-deficient (DLD1, HT-29, LS180) and APC WT (RKO) CRC lines were identified (FIG. 5A) and shRNA-mediated depletion of TDO2 in DLD1, RKO, LS180, and HT29 lines was performed using two independent effective shRNAs (#3 and #6) (FIG. 5B). TDO2 depletion in APC WT RKO cells had no effect on colony formation while APC-deficient DLD1 cells showed markedly reduced colony formation. Moreover, CRISPR/Cas9-mediated mutational inactivation of APC in RKO cells resulted in sensitivity to TDO2 depletion, i.e., decreased colony formation relative to parental APC WT RKO cells (FIG. 5C). In addition, TDO2-specific inhibitor 680C91 (Pilotte et al., 2012) impaired the growth and survival of APC deficient cell lines but not APC intact cells including CCD-841-CoN cells, which are normal colon epithelial cells (FIG. 5D). The murine CRC cell line MC38 and its isogenic APC-null control also demonstrated that APC depleted cells specifically require TDO2 for cell growth and have higher sensitivity to TDO2 inhibition (FIGS. 4A-4C). Intestinal organoids isolated from APCmin mice were infected with inducible shTDO2 also showed increased cell death and impaired growth upon TDO2 depletion (FIGS. 4D and 4E; FIG. 5I).

Decreased growth of orthotopic tumors by TDO2 depletion was observed in APC-null RKO cells in immune deficient NSG mice (FIG. 5E). DLD-1 cell lines expressing shTDO2 exhibited reduced tumor growth which was rescued by enforced expression of a hairpin-resistant TDO2 ORF (FIG. 5F). Pathological analysis of TDO2-depleted tumor tissues revealed decreased cancer cell proliferation (Ki67) and increased apoptosis (cleaved Caspase-3), indicating that TDO2 serves to drive cancer cell-intrinsic hallmarks (FIG. 5G; FIG. 5H).

To explore TDO2 actions in immune competent C57BL/6J mice, APC-null and parental MC38 cells were engineered with an inducible shTDO2 system which revealed impaired growth and increased apoptosis only with the TDO2 depleted APC-KO MC38 cells, but not APC intact controls (FIGS. 4F and 4G). TDO2 depleted APC-KO MC38 cells showed reduced tumor growth associated with decreased Ki67, increased cleaved caspase-3 and increased survival (FIGS. 4H-4L). Orthotopic tumors in NSG mice with APC-KO MC38 cells exhibited similar survival to orthotopic C57BL/6J mice but showed shorter survival benefit with TDO2 depletion when compared to immune competent mice, pointing to an important role for TDO2 in regulating tumor immunity in APC null cancers (FIG. 4M).

The inventors synthesized the TDO2 inhibitor PF06845102/EOS200809 (“TDO2i”), which has been documented to inhibit TDO2 activity in vivo (Schramme et al., Cancer Immunol Res. 2020 January; 8(1):32-45; incorporated herein by reference in its entirety) and administrated the drug by oral gavage to mice bearing APC-WT or APC-KO MC38 orthotopic tumors. As shown in FIG. 4N, the TDO2 inhibitor specifically improved the survival of mice bearing APC-KO MC38 tumors and had no effect on the survival of mice bearing APC-WT MC38 tumors. These findings strongly confirm the context specific role of TDO2 in APC-deficient tumors. Moreover, toxicology analyses showed that the TDO2 inhibitor was well tolerated with no signs of liver toxicity or weight loss. Finally, as shown in FIG. 4O, the IDO inhibitor, Epacadostat (“Epa”), did not exhibit anti-tumor effects in mice bearing either MC38 APC-WT or APC-KO CRC orthotopic tumors. TDO2i treated tumors exhibited decreased Ki67, increased cleaved caspase-3, and decreased M2-like macrophage infiltration (F4/80 and CD163), the latter confirming the role of TDO2 in tumor associated macrophage biology (FIG. 4P).

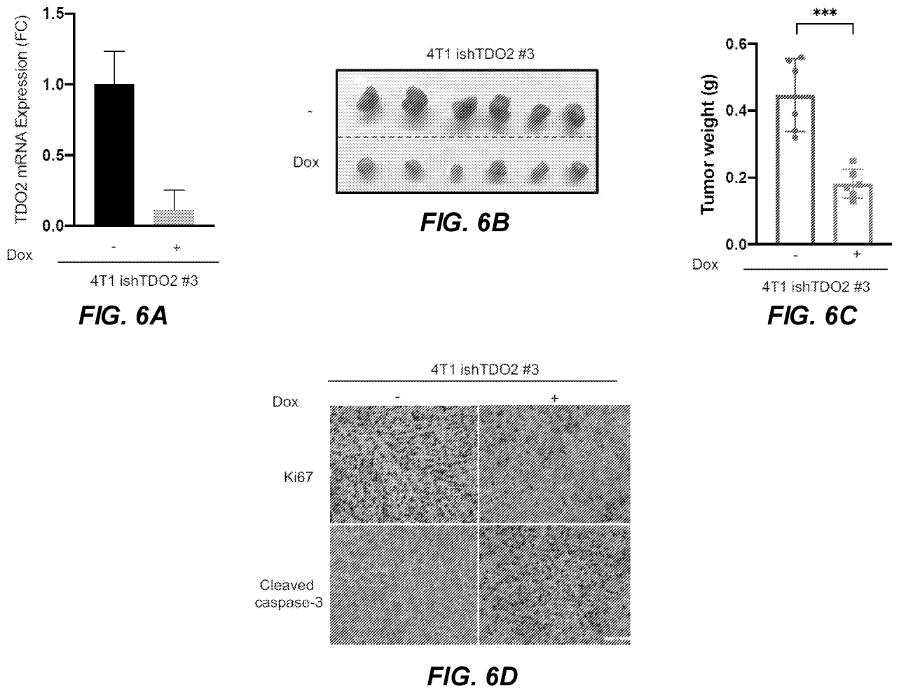

In addition, the Wnt signaling-dependent breast cancer cell line 4T1 (Ganesh et al., 2018) with inducible shTDO2 showed reduced orthotopic tumor growth upon TDO2 depletion (FIGS. 6A-6D). Collectively, these data indicate that TDO2 plays an essential role in supporting cancer cell survival and tumor growth specifically in the setting of APC loss and WNT pathway activation.

### Example 4—TDO2-Kyn-AhR Axis Supports Tumorigenic Potential of APC-Mutant CRC Cancer Cells

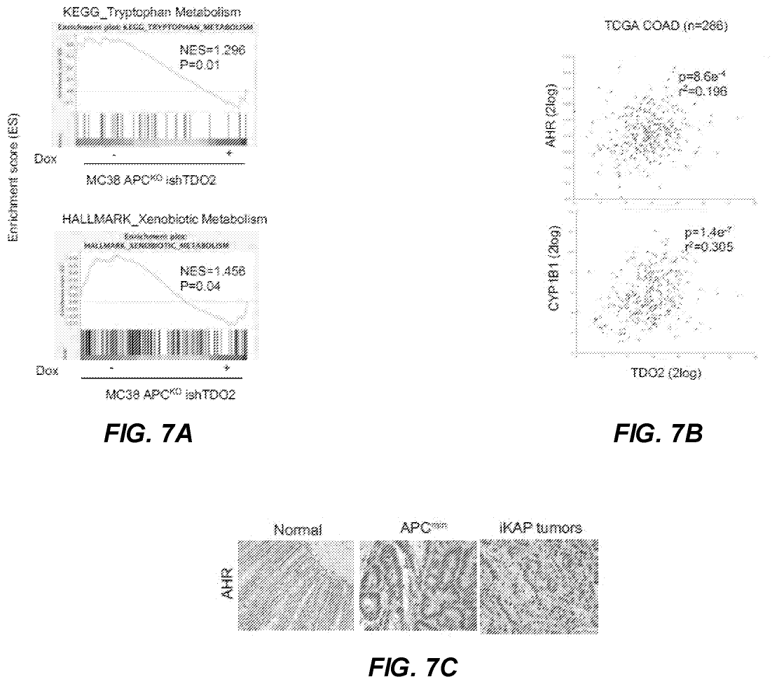

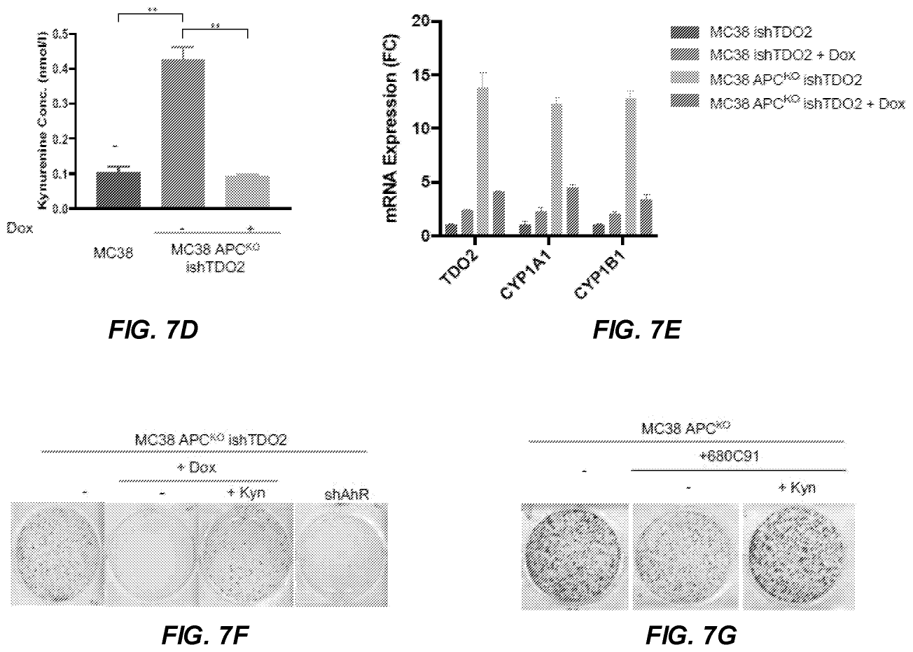

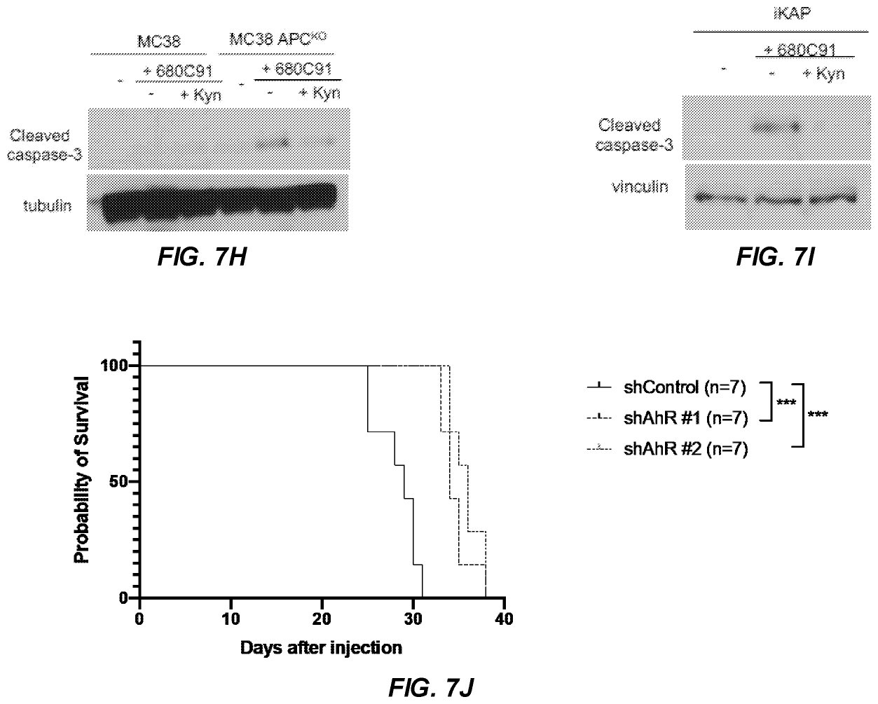

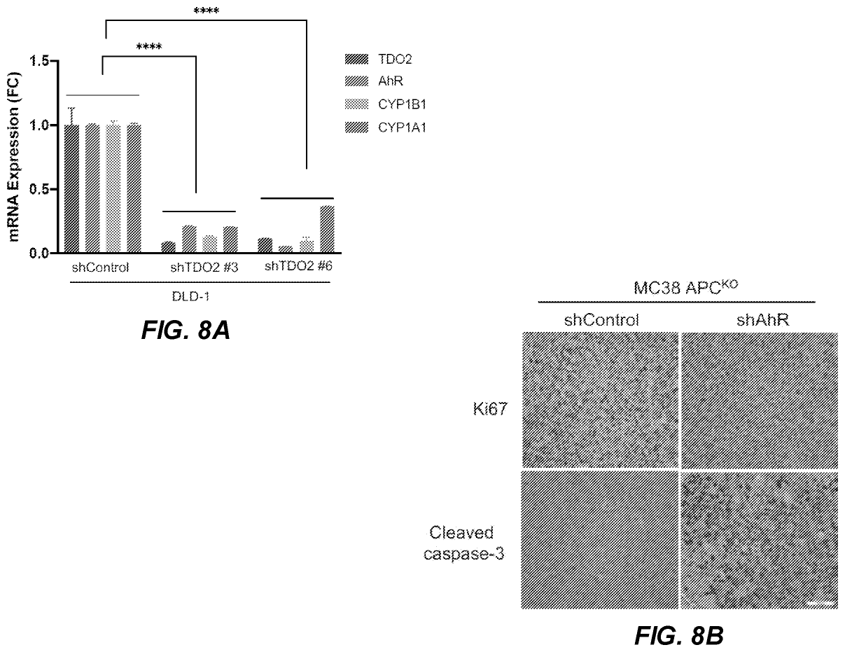

TDO2 metabolizes tryptophan (Trp) to produce kynurenine (Kyn) which in turn activates AhR to upregulate genes governing myriad cellular functions. Gene Set Enrichment Analysis (GSEA) of isogenic APC-KO and APC-WT MC38 cell lines showed that Dox-induction of shTDO2 decreased signatures of tryptophan metabolism as well as xenobiotic metabolism, patterns consistent with the main functions of AhR pathway (FIG. 7A). Correspondingly, expression of AhR and its target gene CYP1B1 correlated positively with TDO2 levels in TCGA COAD dataset (FIG. 7B). Polyps from APCmin mice and iKAP tumors also showed elevated AhR (FIG. 7C). Moreover, APC-KP connection was verified in the APC-KO MC38 model system via ELISA which documented elevated Kyn secretion relative to APC-WT controls and reduced Kyn levels upon TDO2 depletion (FIG. 7D). Finally, gene expression analysis showed down-regulation of AhR expression and its downstream genes upon TDO2 depletion specifically in APC-KO MC38 and DLD1 cell lines (FIG. 7E; FIG. 8A).

To assess the roles of TDO2-Kyn-AhR axis in cancer relevant processes, colony formation assays were performed using the APC-KO MC38 ishTDO2 cell lines and APC-KO MC38 shAhR cell lines. In APC-KO MC38 cells, TDO2 and AhR depletion showed reduced colony formation which was rescued by Kyn treatment (FIG. 7F). Similarly, using APC-KO MC38 cells, increased cell death was documented with 680C91 treatment, as indicated by caspase-3 assay, which was rescued by Kyn supplementation (FIGS. 7G and 7H). As another confirmatory model, the iKAP model system were utilized and showed the decreased cell death by treating Kyn (FIG. 7I). Moreover, mice injected with APC-KO MC38 cells exhibited increased survival upon AhR depletion and tumors exhibited decreased Ki67 and elevated cleaved Caspase-3 (FIG. 7J; FIG. 8B). Thus, the TDO2-Kyn-Ahr axis supports APC-null cancer cell proliferation and survival and its tumorigenic potential.

### Example 5—TDO2 Depletion Inhibits Infiltration of Tumor Associated Macrophages

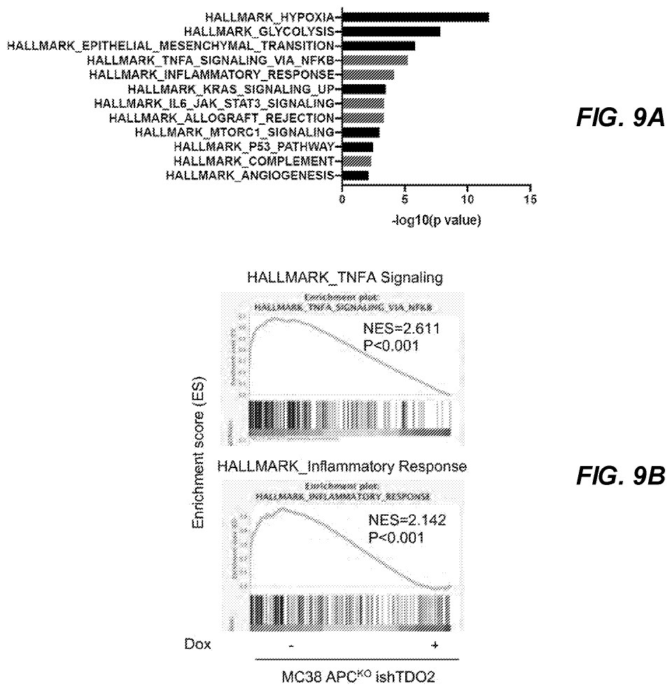

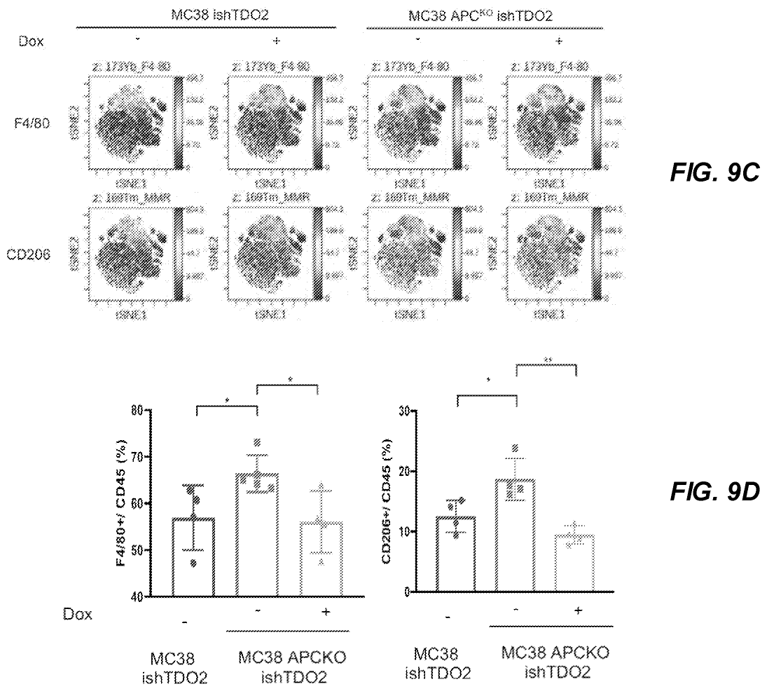

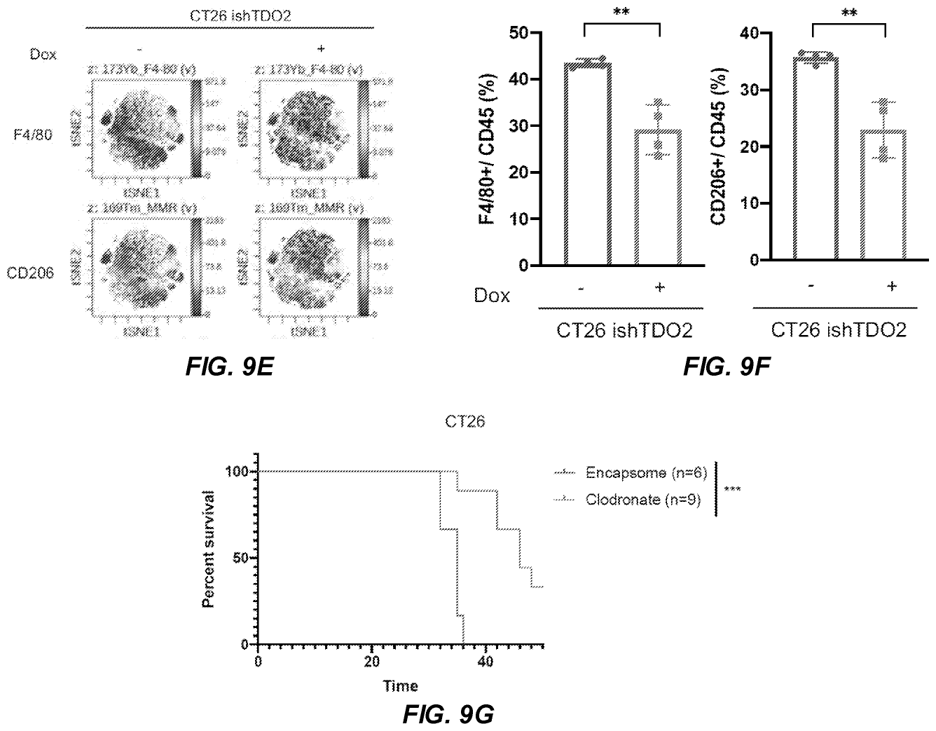

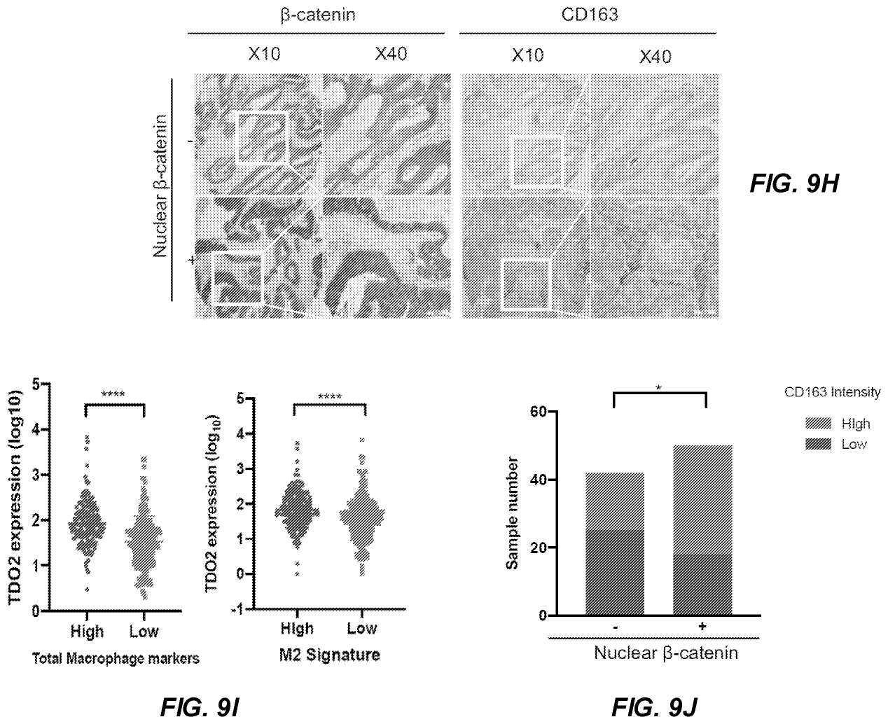

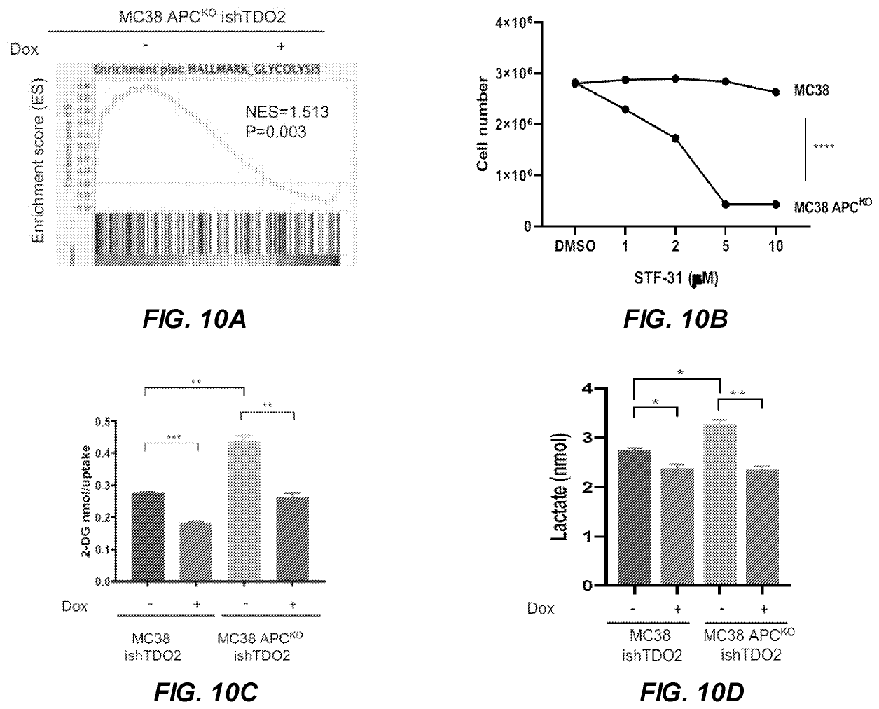

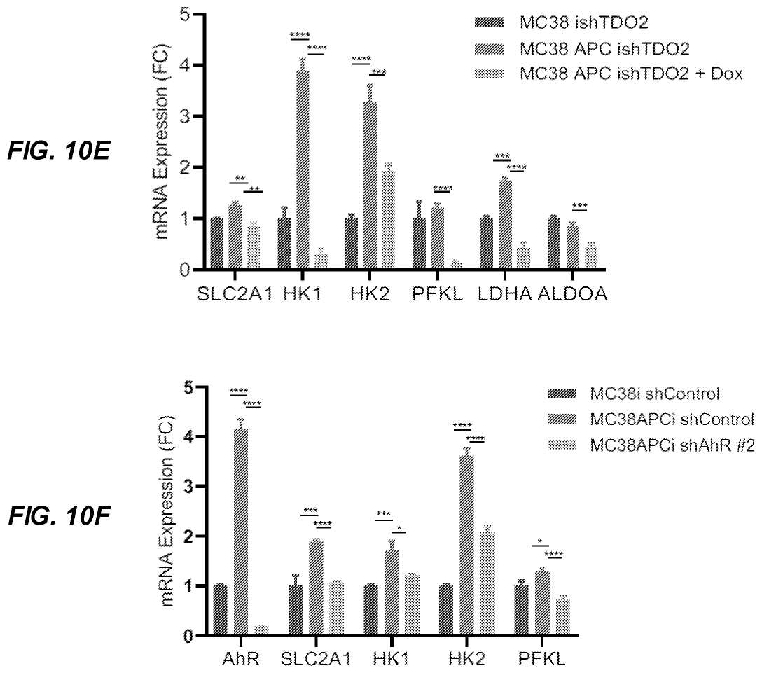

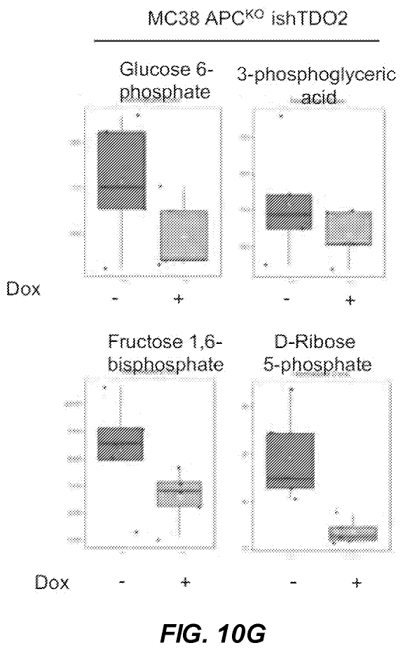

To assess cancer hallmarks impacted by APC deletion and TDO2 depletion in CRC, GSEA analysis of RNA-seq data from APC-KO MC38 ishTDO2 cell lines and microarray data from derivative tumors was performed. Consistent with known cancer cell intrinsic functions of the APC-WNT pathway (Pate et al., 2014), hypoxia and glycolysis pathways were up-regulated in APC-KO cells (FIG. 9A; FIG. 10A). APC-KO MC38 cells exhibited higher sensitivity to GLUT1 inhibitor STF-31 than APC-WT MC38 cells (FIG. 10B) and increased glucose uptake and lactate secretion, which were reversed by TDO2 depletion (FIGS. 10C and 10D). RT-PCR analysis confirmed up-regulation of key glycolysis genes such as SLC2A1, HK1/2, and PFKL, and these genes were down-regulated upon TDO2 or AhR depletion (FIGS. 10E and 10F). Metabolite analysis of cell lysates and conditioned media from APC-KO MC38 cells showed decreased levels of glycolysis pathway-related metabolites upon TDO2 depletion (FIG. 10G). These data denote glycolysis pathways as a key cancer cell intrinsic process regulated by TDO2-AhR signaling.

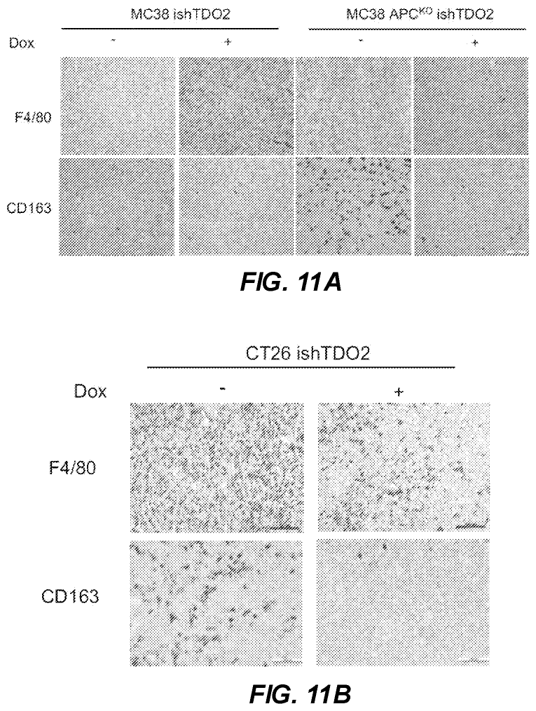

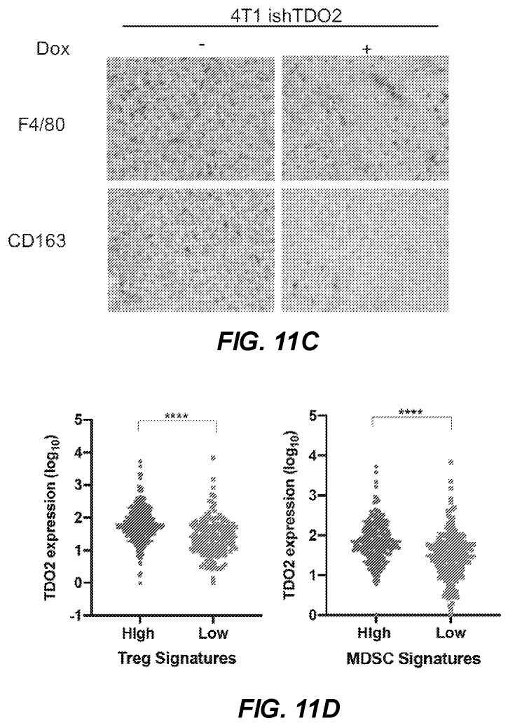

In addition to these cancer cell-intrinsic pathways, APC status (APC-KO versus APC-WT MC38) or TDO2 depletion in APC deficient cell lines and tumors resulted in prominent representation of immune signaling signatures such as TNFA signaling, inflammatory response, IL-6_JAK_STAT, allograph rejection, and complement (FIGS. 9A and 9B). These in silico observations prompted immunoprofiling of orthotopic tumors established with isogenic APC-KO and APC-WT MC38 cells with and without TDO2 depletion. viSNE plots of CyTOF data showed that APC KO resulted in a significant increase in macrophage abundance, while TDO2 depletion decreased macrophage abundance (FIG. 9C). Quantification data of F4/80 positive immune cells and CD206high M2 populations in CD45 positive cells confirmed enrichment of macrophages in APC-KO tumors and their reduction upon TDO2 depletion (FIG. 9D). Immunohistochemistry staining of CD163 in these tumors aligned with the aforementioned CyTOF data (FIG. 11A). Tumors established with the WNT-active CRC cell line CT26 and breast cancer cell line 4T1 also exhibited decreased macrophage populations upon TDO2 depletion (FIGS. 9E and 9F; FIGS. 11B and 11C). Balb/C mice injected with CT26 ishTDO2 cell lines showed prolonged survival when macrophages were depleted by treating clodronate liposomes, underscoring the importance of tumor associated macrophages in this model system (FIG. 9G).

To further corroborate the TDO2-mediated TME modulation, TCGA CRC datasets were examined for expression of macrophage (total and M2) as well as Treg and MDSC markers, revealing strong positive correlations between WNT activation and TDO2 expression (FIG. 9H; FIG. 11D). In addition, the WNT-macrophage correlation was further validated by CRC tumor microarrays (TMAs) analyses which showed that cancer cells with nuclear beta-catenin signal exhibited higher CD163 expression in the TME (FIGS. 9I and 9J). Together, these findings support the model that WNT pathway-activated upregulation of TDO2 activates the AhR network which recruits immune suppressive tumor associated macrophages into the TME.

### Example 6—TDO2-AhR-CXCL5 Promotes Tumor Growth by Recruiting Tumor Associated Macrophages (TAMs) into the CRC TME

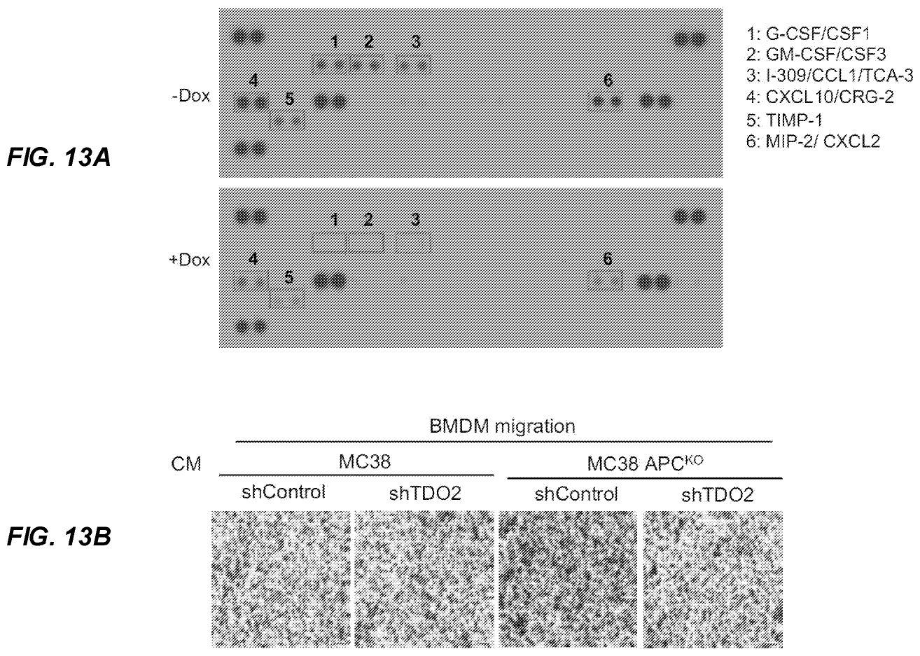

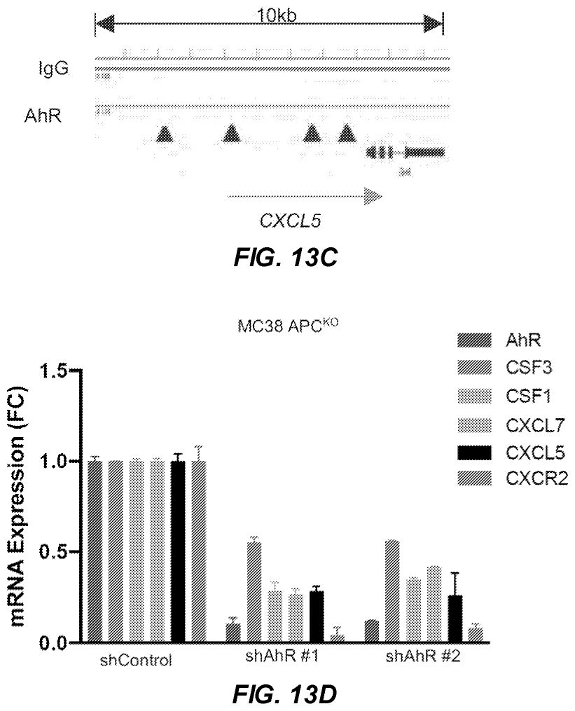

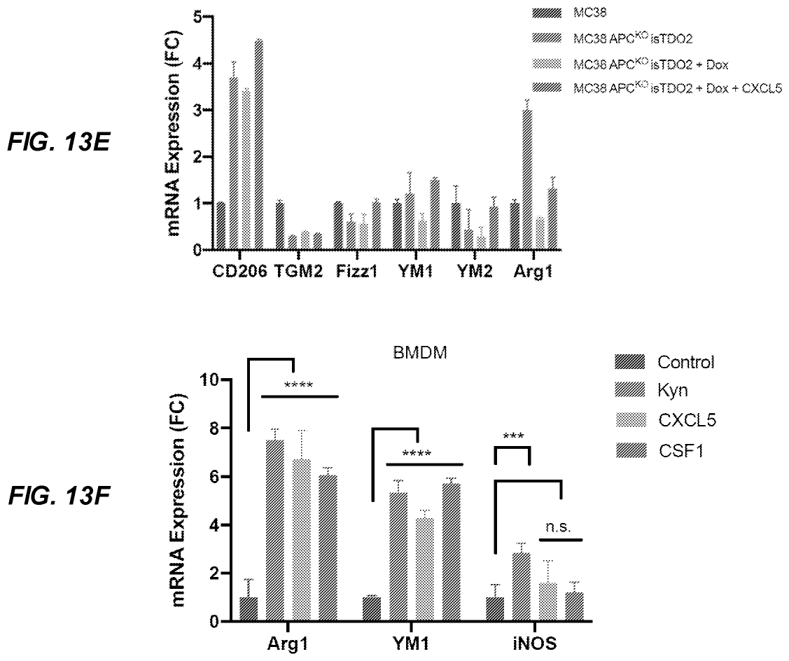

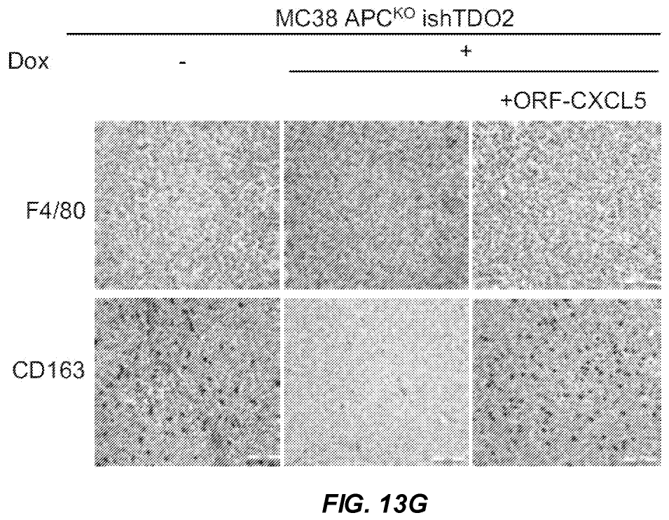

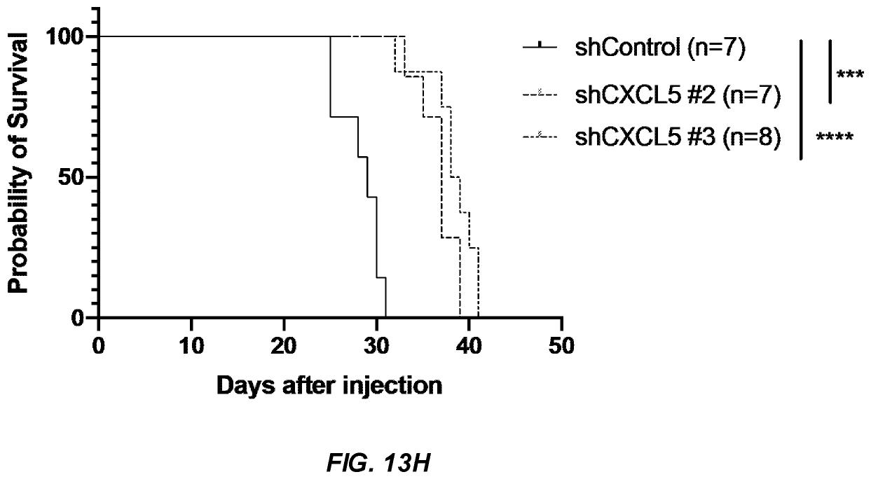

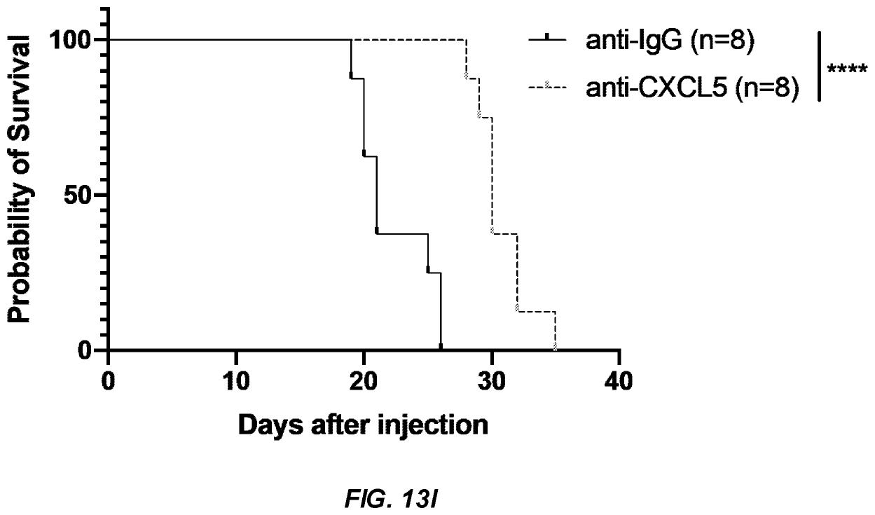

To identify WNT-TDO2-AhR pathway targets that may recruit TAMs, cytokine array profiling of conditioned media (CM) from APC-KO MC38 ishTDO2 cells was performed. TDO2 depletion reduced secretion of classical macrophage cytokines including G-CSF, GM-CSF, CXCL2 (FIG. 13A) and other cytokines (see below). Correspondingly, transwell migration assays using bone marrow-derived macrophages (BMDM) showed that CM from APC-KO MC38 ishTDO2 cultures increased macrophage migration which was nullified upon TDO2 depletion (FIG. 13B).

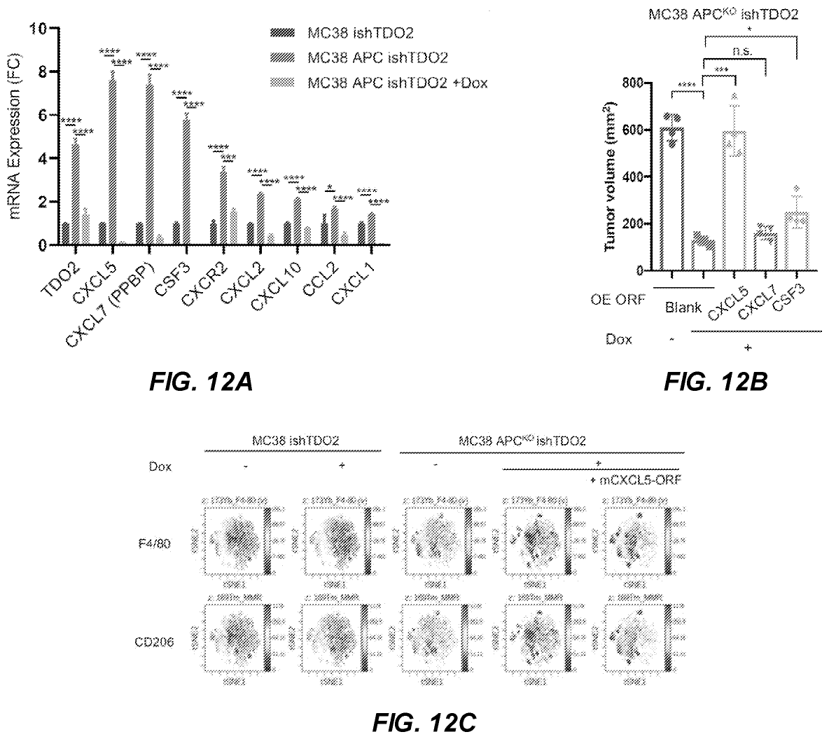

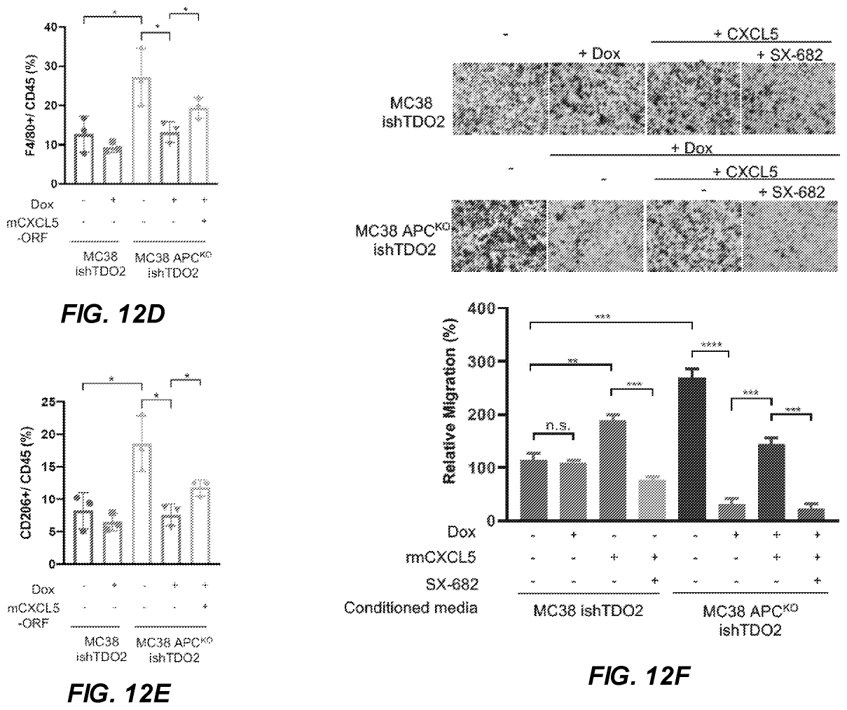

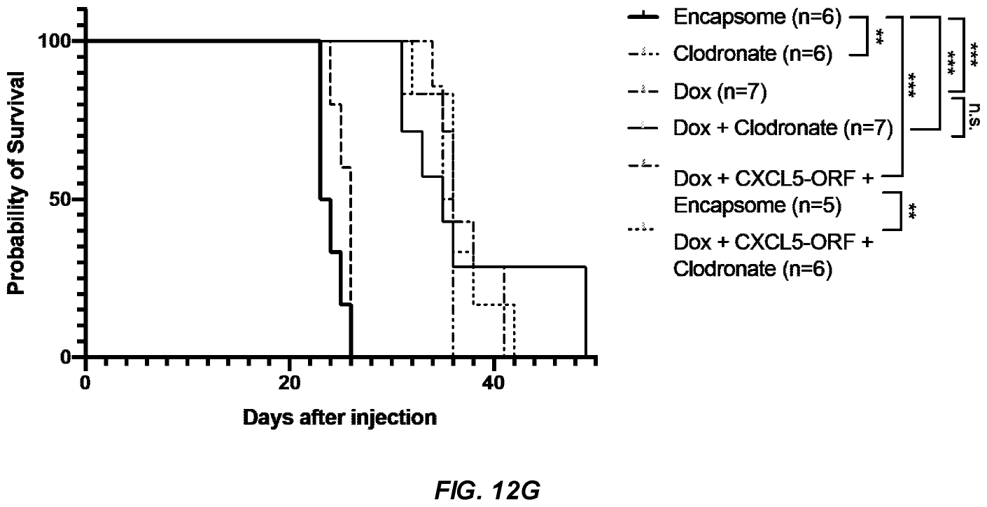

Next, to more fully vet most highly regulated cytokines, the top ranked genes in the RNA-seq dataset were identified and qRT-PCR validated. CXCL5, CXCL7 (PPBP), CSF3 (G-CSF), CXCR2, CXCL2, CXCL10, CCL2, and CXCL1 showed the most significant expression changes associated APC deletion or TDO2 depletion (FIG. 12A). CHIP-seq analysis of AhR gene targets in APC-KO MC38 cells showed direct AhR binding to these genes (FIG. 13C) and AhR depletion by shRNA in APC-KO MC38 cells inhibited the expression of these target genes (FIG. 13D).

To further identify the target cytokines of TDO2, cell lines that express ORFs of the top three genes (CXCL5, CXCL7 (PPBP), and CSF3) from RNA-seq data were generated in APC-KO MC38 ishTDO2 cells and tumor growth was monitored to identify genes that rescue the impaired proliferation by TDO2 knockdown. Enforced expression of CXCL5, which showed highest fold changes, most significantly rescued the decreased tumor growth mediated by TDO2 depletion (FIG. 12B). CyTOF analysis of CXCL5-overexpressing APC-KO tumors showed increased macrophages in the presence of shTDO2 (FIGS. 12C-12E).

Migration assays showed rescue of macrophage recruitment by supplementing CXCL5 to the CM from APC-KO TDO2 depleted MC38 cells whereas co-treatment of CXCR1/2 inhibitor, to which CXCL5 binds, abrogated the rescue by CXCL5 supplementation (FIG. 12F). Conditioned media from APC-KO MC38 cells upregulated several M2 macrophage markers in Raw264.7 cells, which were decreased by TDO2 knockdown and rescued by CXCL5 supplementation (FIG. 13E). Similarly, when BMDMs were co-cultured with Kyn or CXCL5, M2 macrophage markers increased, suggesting potential roles of TDO2-AhR axis in TAM polarization (FIG. 13F).

Immunohistochemical analysis of macrophage markers showed increased infiltration of macrophages in tumors with enforced CXCL5 expression (FIG. 13G). Finally, xenograft mice with APC-KO MC38 cells showed increased survival by TDO2 or macrophage depletion. CXCL5 overexpression in APC-KO MC38 cell lines significantly shortened the survival of mice, which was reversed by depleting macrophages (FIG. 12G). APC-KO MC38 cells with shCXCL5 or treated with anti-CXCL5 neutralizing antibody also showed prolong survival (FIGS. 13H and 13I).

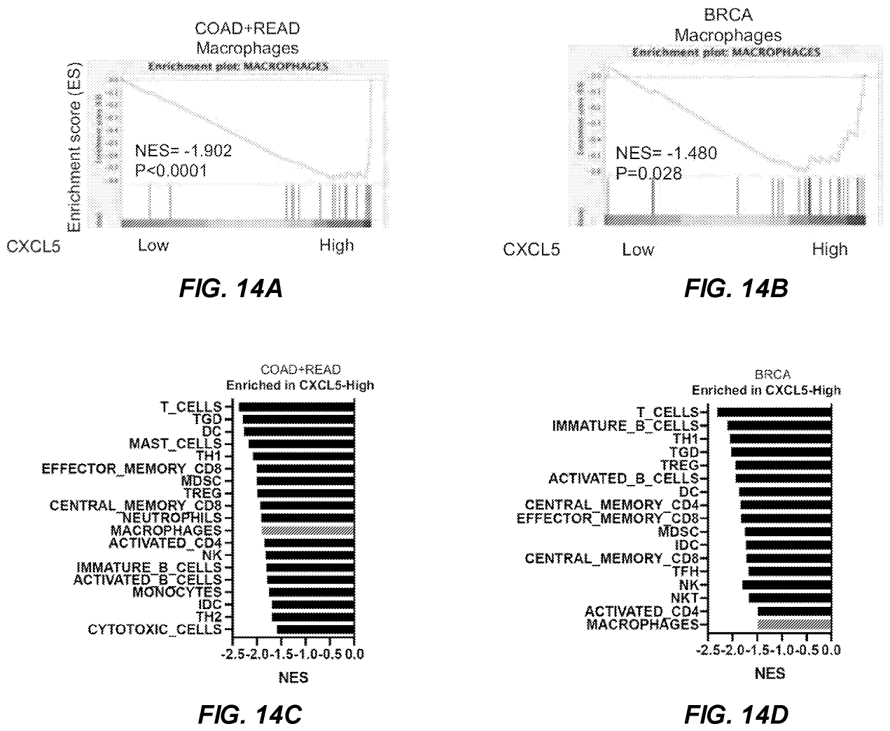

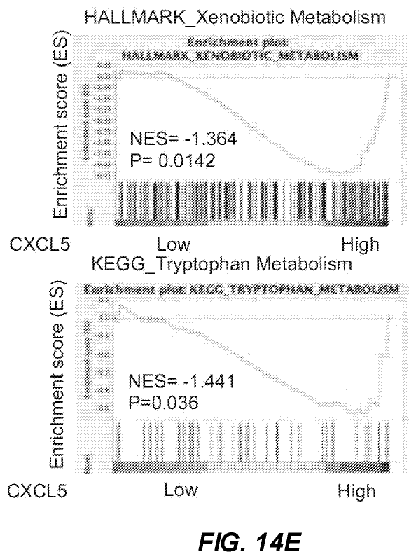

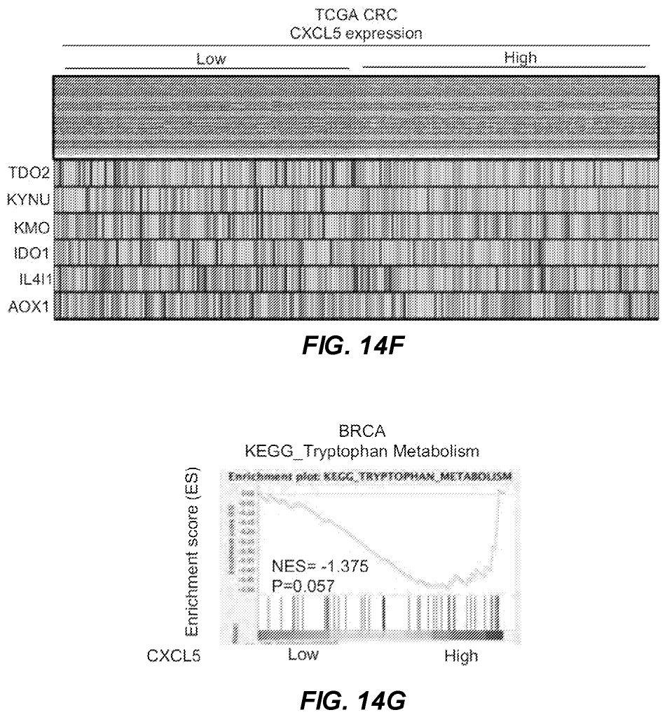

To further validate the relationship between the TDO2-AhR-CXCL5 axis and macrophage infiltration, TCGA CRC (COAD and READ) and BRCA datasets were clustered based on CXCL5 expression and analyzed for immune populations that are correlated and found that macrophages are upregulated in CXCL5-high group (FIGS. 14A-14D). In addition, CXCL5 expression was significantly correlated with increased tryptophan metabolism (TDO2 as top pathway signature gene) and xenobiotic metabolism in TCGA CRC and BRCA (trend) (FIGS. 14E-14G). Together, these data demonstrate that upregulation of CXCL5 by the TDO2-AhR pathway drives the recruitment of macrophages into the TME to promotes tumor growth and the neutralization of the TDO2-AhR-CXCL5 pathway can inhibit tumor growth.

### Example 7—GAS6 is Upregulated by CXCL5 in Macrophages and Promotes Cell Growth

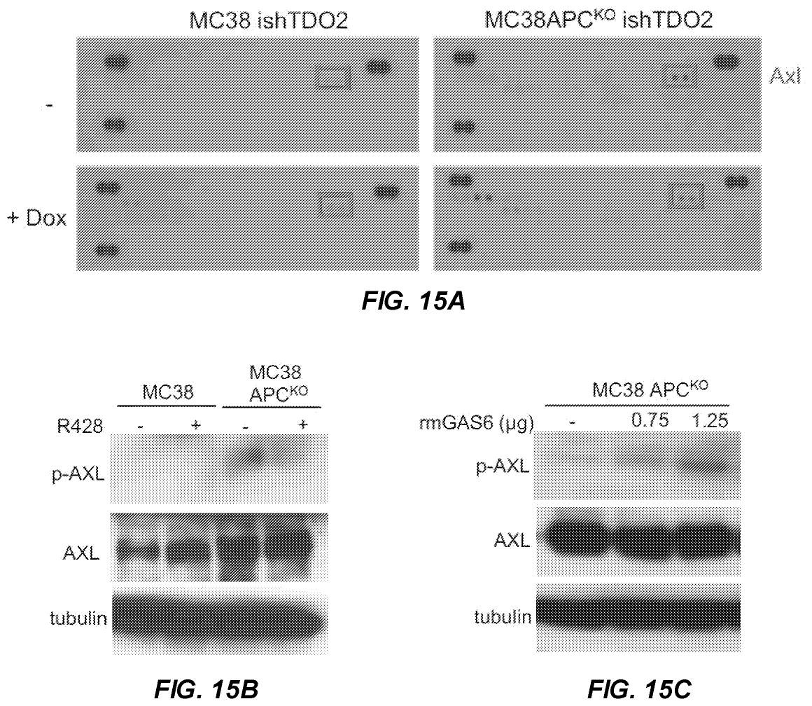

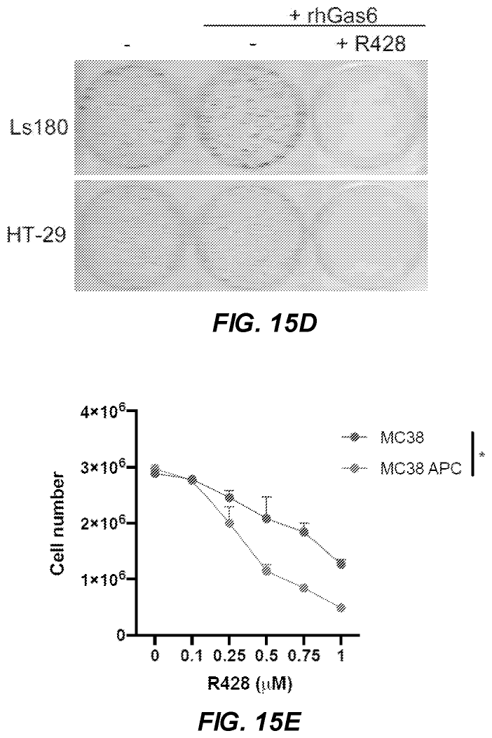

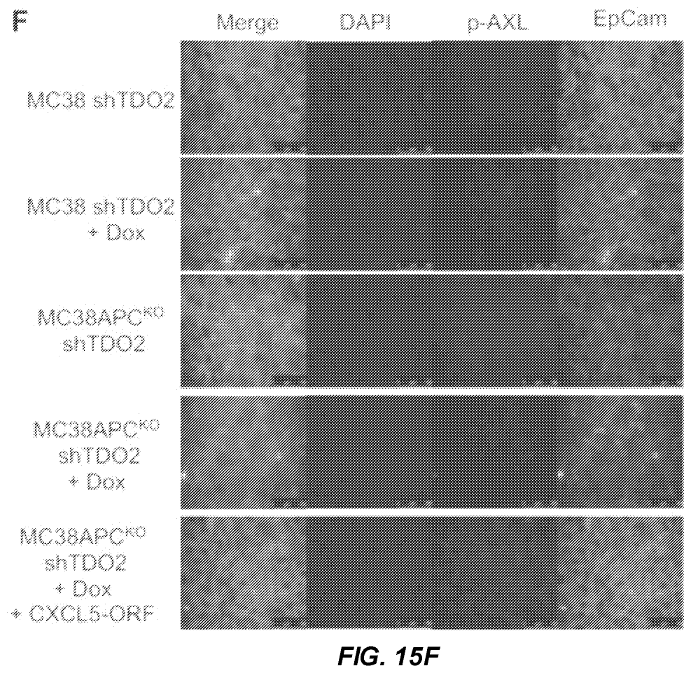

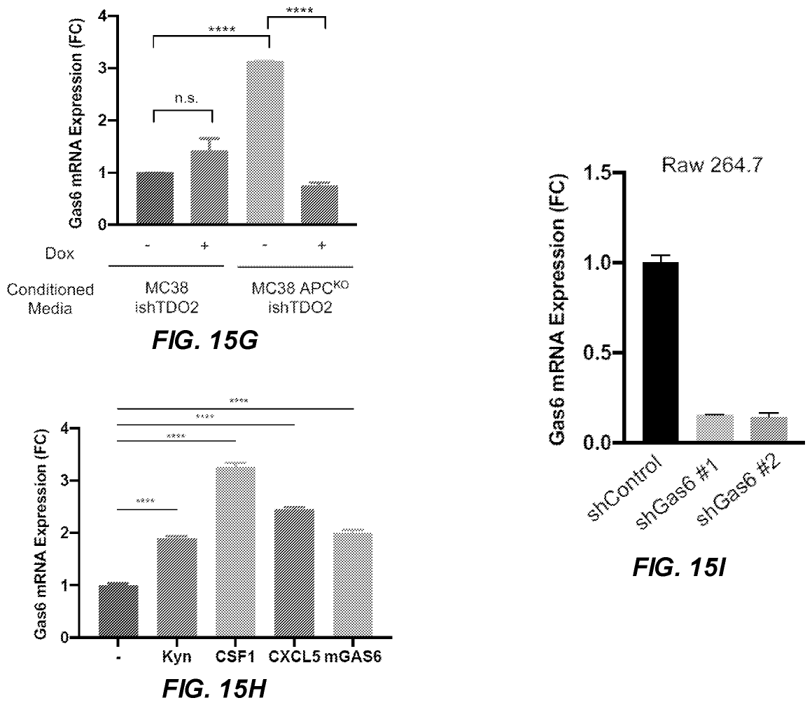

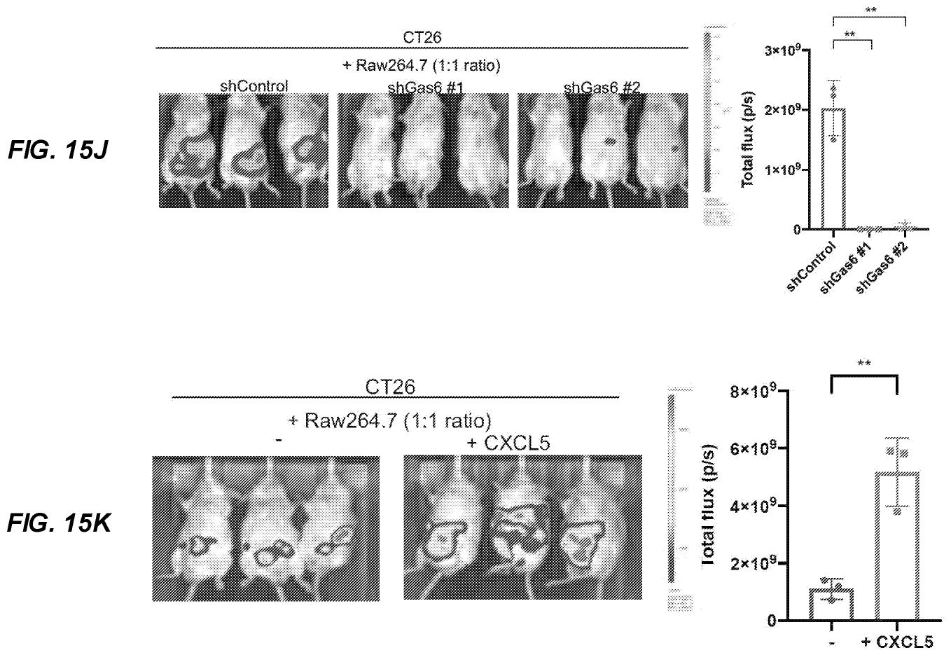

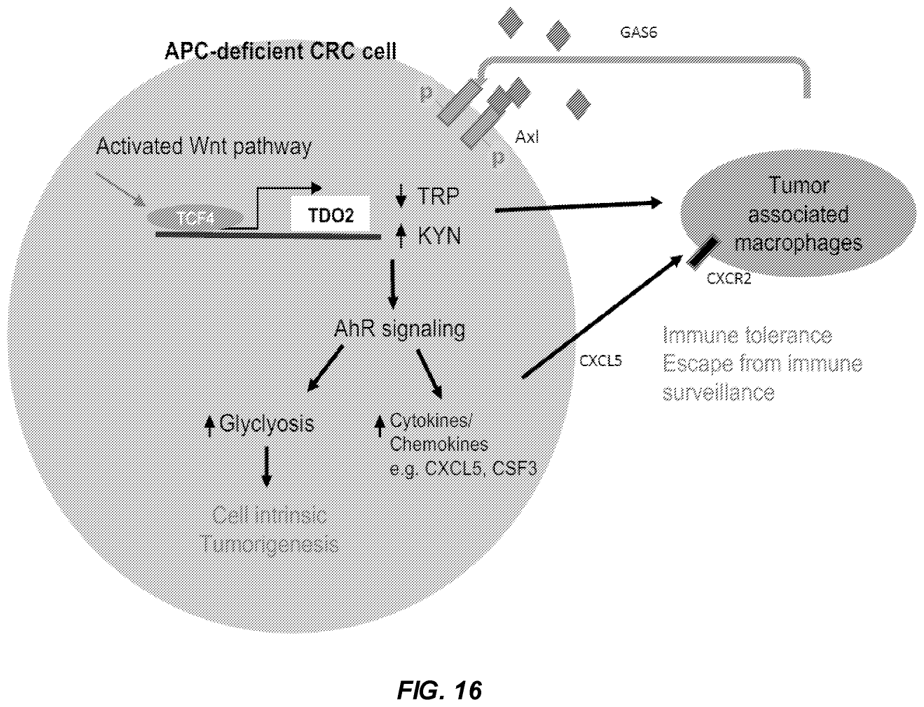

Receptor tyrosine kinase phosphoarray analysis was conducted, revealing prominent up-regulation of phosphorylated Axl in APC-KO cells which was decreased upon TDO2 depletion only in the APC-KO cell lines (FIG. 15A).

Gas6 is a ligand of Axl and is secreted mainly by tumor-associated macrophages in the TME (Loges et al., 2010), raising the possibility that macrophage-derived Gas6 might activate Axl to support the growth of APC-deficient cancer cells. To assess this possibility, APC-KO cells were validated to have increased total Axl and Axl phosphorylation and Axl inhibitor R428 was shown to reduce Axl phosphorylation (FIG. 15B). Reciprocally, mouse recombinant Gas6 (rmGas6) activated Axl (FIG. 15C) and promoted cancer cell growth which was reversed by R428 treatment in vitro (FIG. 15D). Moreover, APC-deficient cells exhibited increased sensitivity to R428 when compared to APC-intact MC38 cells (FIG. 15E). In addition, orthotopic tumors established with APC-KO MC38 cells showed increased phosphorylated Axl in the tumor cells (indicated by EpCam staining) which was reduced by TDO2 depletion. These orthotopic tumors also showed enforced expression of CXCL5 in TDO2-depleted APC-KO MC38 cells rescued the phosphorylated Axl (FIG. 15F).

To evaluate whether secreted factors from cancer cells might upregulate Gas6 expression in macrophages, BMDMs were incubated with conditioned media from MC38 cell lines. Conditioned media from APC-KO MC38 cell lines significantly increased the Gas6 mRNA levels in macrophages compared to APC-WT cell lines and APC-KO MC38 cells with TDO2 depletion (FIG. 15G). In addition, while CSF1 is known to upregulate Gas6 in macrophages, Kyn and CXCL5 also increased Gas6 expression in BMDM as well as the Gas6 itself, suggesting a positive feedback loop. (FIG. 15H). Finally, to validate the roles of Gas6 in promoting tumor growth in vivo, CT26 cell lines and two independent Raw 264.7 shGas6 cell lines were co-injected into syngeneic Balb/CJ mice (FIG. 15I). Mice injected with Gas6 depleted macrophages showed reduced tumor growth (FIG. 15J) and macrophages pre-treated with CXCL5 promoted the growth of CT26 significantly (FIG. 15K). Collectively, these data revealed a symbiotic relationship between cancer cells and macrophages which is mediated in part by a CXCL5-Gas6-Axl circuit.

### Example 8—Materials and Methods Related to Certain Studies Described in Examples 1-7

### Materials

### Mice

Mice were grouped by 5 animals in large plastic cages and were maintained under pathogen-free conditions. All animal studies were performed with the approval of MD Anderson Cancer Center's Institutional Animal Care and Use Committee (IACUC). NSG (NOD.Cg-Prkdc^{scid} Il2rg^{tm1Wjl}/SzJ), C57BL/J6J, and BALB/cJ mice were purchased from Jackson laboratory (Stock No: 005557, 000664, 000651). Colorectal orthotopic xenograft tumor models were established by following protocol (Tseng et al., 2007, JoVE).

### Cells

The CRC cell lines MC38 and its isogenic cells as well as BMDM and 293T cells were cultured in Dulbecco's Modified Eagle's Medium (DMEM). CCD-841-CoN, RKO, HT-29 and LS180 cells were cultured in Eagle's Minimum Essential Medium (EMEM). HT-29 cells were cultured in McCoy's 5A medium. DLD-1, CT26, 4T1 and Raw264.7 macrophage cell line was cultured in RPMI 1640 medium (RPMI). All cell lines were cultured in indicated medium containing 10% Tet System Approved FBS (Clontech) and 100 U/ml ampicillin/penicillin. All human cell lines have been validated through fingerprinting by the MD Anderson Cell Line Core Facility. All cells were confirmed to be mycoplasma-free and maintained at 37° C. and 5% C02. BMDMs from C57BL/6 mice were cultured as previously described (Chen et al., 2017). Conditioned media were collected from treated or untreated cells as indicated after culturing for 24 h in FBS-free culture medium.

### CRISPR-Cas9 Transfection

sgRNA plasmids targeting human APC gene (sc-400374) were purchased from Santa Cruz Biotechnology. For mouse APC gene, a sgRNA target sequence of TTGAGCGTAGTTTCACTCCG was cloned into pCas-Guide-EF1a-GFP plasmids (Origene Technologies, Inc.). Human RKO and mouse MC38 cells were maintained in 6-well plates to a 70-80% confluency in culture media supplemented with 10% heat-inactivated FBS and 100 U/ml ampicillin/penicillin. The plasmids with sgRNA were transiently transfected into using Lipofectamine 2000 according the manufactory protocol. Cells were harvested 72 h later, and GFP-positive cells were sorted into each well of a 96-well plate as single cell by flow cytometry. At day 10 after cell sorting, the grown cell colonies were expanded in 24-well plates. The knock-out of APC gene in each colony was confirmed by RT-PCR and western blot for APC and β-catenin.

### APC^{min} Mouse Organoids

Intestinal polyps from a 18 week-old male APC^{min} mouse were harvested and the cut tissue were treated with complete chelating solution containing 30 mM EDTA for 30 min at 4° C. The tissue pieces were then pipetted gently to dissociate the crypts. These crypts were then seeded in Matrigel (Corning) in the presence of high WNT human organoid media in the presence of ROCK inhibitor Y-27632 for 7-10 days.

### Methods

Gene Stable shRNA Knockdown and Inducible shRNA Knockdown

shRNA hairpins targeting human TDO2 and AhR as well as mouse were used in this study. Five to seven hairpins were screened of each gene and sequences were chosen that reduced protein levels by >70%. These selected hairpins were in the pLKO.1 vector. Recombinant lentiviral particles were produced by transient transfection of plasmids into 293T cells. In brief, 8 μg of the shRNA plasmid, 4 μg of the psPAX2 plasmid, and 2 μg of the pMD2.G plasmid were transfected using Lipofectamine 2000 into 293T cells plated in 100-mm dishes. Viral supernatant was collected 48 h and 72 h after transfection and filtered. Cells were infected twice in 48 h with viral supernatant containing 8 μg/ml polybrene, and then selected using 2 μg/ml puromycin and tested the expression TDO2 and AhR by RT-qPCR.

### Western Blot

Cell lysates were prepared with RIPA lysis buffer (Roche) with protease inhibitor cock Immunoblotting was performed following standard protocol. Antibodies were purchased from the indicated companies.

### Immunohistochemistry and Immunofluorescence

Immunohistochemistry was performed using standard protocol as previously described (Chen et al., 2017; Zhao et al., 2017). In brief, a pressure cooker (95° C. for 30 min followed by 120° C. for 10 s) was used for antigen retrieval using antigen unmasking solution (Vector Laboratories). Antibodies specific to X were used in this study. The human and mouse tumor tissue sections were reviewed and scored. Slides were scanned using Pannoramic 250 Flash III (3DHISTECH Ltd) and images were captured through Pannoramic Viewer software (3DHISTECH Ltd). The studies related to human specimens were approved by the MD Anderson Institutional Review Board.

### Migration Assay

Macrophages (1×10⁴ for Raw264.7 and BMDM) were suspended in serum-free culture medium and seeded into 24-well Transwell inserts (5.0 m). Medium with indicated factors or conditioned media was added to the remaining receiver wells. After 24 h, the migrated macrophages were fixed and stained with crystal violet (0.05%, sigma), and then counted as cells per field of view under microscope or measured as OD590 nm (expressed as migration index) after crystal violet was dissolved.

### Colony Formation Assay

Colorectal cancer cell proliferation in vitro was assayed through colony formation. 1×10³ cells were seeded in each well of 6-well plates and cultured for 5-7 days. At the end point, cells were fixed and stained with 0.5% crystal violet in 25% methanol for 1 hr. These studies were performed in triplicate.

### Mass Cytometry (CyTOF)

CyTOF analysis was performed as described previously (Lao et al., 2019). Briefly tumors were digested and single cells blocked with FcR were incubated with surface antibody. Cells were then incubated with Cell-ID Cisplatin (Fluidigm, Cat #201064) and permeabilized for FOXP3 intracellular staining. For nuclei staining, cells were incubated with Cell-ID Intercalator-Ir (Fluidigm, Cat #201192A) while fixing. Samples were analyzed with a CyTOF instrument (Fluidigm) in the Flow Cytometry and Cellular Imaging Core Facility at MD Anderson Cancer Center. Cell numbers and percentages of each cell populations were analyzed with FlowJo (Tree Star) and GraphPad Prism 6 software. CyTOF data were visualized using a dimensionality reduction method viSNE (Amir el et al., 2013), which was implemented using the Cytobank (Chen and Kotecha, 2014).

### ChIP-Sequencing and ChIP-PCR

ChIP was performed as described previously (Zhao et al., 2017). Briefly, chromatin from PFA-fixed cells were cross-linked using 1% PFA for 10 min and then reactions were quenched using 0.125 M glycine (5 min) at room temperature. Cells were lysed with ChIP lysis buffer [10 mM Tris-HCl (pH 8.0), 140 mM NaCl, 1 mM EDTA (pH 8.0), 1% Triton X-100, 0.2% SDS and 0.1% deoxycholic acid] for 30 min on ice. Chromatin fragmentation was performed using a Diagenode BioruptorPico sonicator (30 s on and 30 s off, 45 cycles). Solubilized chromatin was then incubated with the appropriate mixture of antibody and Dynabeads (Life Technologies) overnight. Immune complexes were then washed with RIPA buffer (three times), once with RIPA-500 (RIPA with 500 mM NaCl), and once with LiCl wash buffer [10 mM Tris-HCl (pH 8.0), 1 mM EDTA (pH 8.0), 250 mM LiCl, 0.5% NP-40 and 0.5% deoxycholic acid]. Elution and reverse-crosslinking were performed in direct elution buffer [10 mM Tris-Cl (pH 8.0), 5 mM EDTA, 300 mM NaCl, 0.5% SDS] containing proteinase K (20 mg/ml) at 65° C. overnight. Eluted DNA was purified using AMPure beads (Beckman-Coulter), which then was used to generate libraries using NEBNext Ultra DNA Library kit (E7370), or to perform qPCR. Sequencing was performed using an Illumina HiSeq 2500 instrument to generate dataset.

mRNA Expression Analysis and Microarray

RNA was isolated from cells using Trizol (Invitrogen) following the manufacturer's instructions. RNA (200-500 ng) was first treated with RNase-free DNase I using the DNA-free kit (Ambion) to remove all genomic DNA, and then reverse-transcribed into cDNA using an ABI High Capacity cDNA RT Kit (Invitrogen). The cDNA was analyzed using real-time quantitative PCR (SYBR Green, Invitrogen) with an Applied Biosystems 7900 Sequence Detection System. Each reaction was performed in triplicate. The expression of each gene was normalized by that of mouse Actin or human GAPDH.

RNA was isolated from cells using TRIzol Reagent (Invitrogen; #15596-026) according to the manufacturer's instructions. RNA was then further purified with RNeasy (QIAGEN) and then was reverse-transcribed into cDNA using the SuperScript III First-Strand Synthesis System (Invitrogen). RT-qPCR was performed to analyze the expression of targeted genes using the SYBR Green PCR Master Mix (Applied Biosystems). Microarray analysis was performed on RNA prepared from APC-WT and APC-KO MC38 ishTDO2 tumors with and without Doxycyclin treatment (biological triplicates for control and APC-KO MC38 tumors) at the MD Anderson Microarray Core facility using the GeneChip Mouse Clariom D array (Affymetrix) to generate dataset. Genes that were differentially expressed between control and APC-depleted MC38 cells were subjected to gene set enrichment analysis (GSEA). The raw data were processed and analyzed by GenePattern using Transcriptome Analysis Console.

### Quantification and Statistical Analysis

All statistical analyses were performed with Student's t-test and represented as mean±SD. The analysis of TAM IHC staining for the correlation among nuclear β-catenin, TDO2, and CD163 was performed using the chi-squared test. Mouse survival analysis was performed using Log-rank (Mantel-Cox) test (GraphPad Prism 7). The p values were designated as: *, p<0.05; **, p<0.01 and ***, p<0.001; n.s. non-significant (p>0.05).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Badawy, A. A. (2017). Kynurenine Pathway of Tryptophan Metabolism: Regulatory and Functional Aspects. Int J Tryptophan Res 10, 1178646917691938.
Boutin, A. T., Liao, W. T., Wang, M., Hwang, S. S., Karpinets, T. V., Cheung, H., Chu, G. C., Jiang, S., Hu, J., Chang, K., et al. (2017). Oncogenic Kras drives invasion and maintains metastases in colorectal cancer. Genes Dev 31, 370-382.
Chen, P., Zhao, D., Li, J., Liang, X., Li, J., Chang, A., Henry, V. K., Lan, Z., Spring, D. J., Rao, G., et al. (2019). Symbiotic Macrophage-Glioma Cell Interactions Reveal Synthetic Lethality in PTEN-Null Glioma. Cancer Cell 35, 868-884 e866.
Chen, P., Zuo, H., Xiong, H., Kolar, M. J., Chu, Q., Saghatelian, A., Siegwart, D. J., and Wan, Y. (2017). Gpr132 sensing of lactate mediates tumor-macrophage interplay to promote breast cancer metastasis. Proceedings of the National Academy of Sciences of the United States of America 114, 580-585.
D'Amato, N.C., Rogers, T. J., Gordon, M. A., Greene, L. I., Cochrane, D. R., Spoelstra, N. S., Nemkov, T. G., D'Alessandro, A., Hansen, K. C., and Richer, J. K. (2015). A TDO2-AhR signaling axis facilitates anoikis resistance and metastasis in triple-negative breast cancer. Cancer Res 75, 4651-4664.
Dow, L. E., O'Rourke, K. P., Simon, J., Tschaharganeh, D. F., van Es, J. H., Clevers, H., and Lowe, S. W. (2015). Apc Restoration Promotes Cellular Differentiation and Reestablishes Crypt Homeostasis in Colorectal Cancer. Cell 161, 1539-1552.
Fang, D. C., Luo, Y. H., Yang, S. M., Li, X. A., Ling, X. L., and Fang, L. (2002). Mutation analysis of APC gene in gastric cancer with microsatellite instability. World J Gastroenterol 8, 787-791.
Furuuchi, K., Tada, M., Yamada, H., Kataoka, A., Furuuchi, N., Hamada, J., Takahashi, M., Todo, S., and Moriuchi, T. (2000). Somatic mutations of the APC gene in primary breast cancers. Am J Pathol 156, 1997-2005.
Gabriely, G., Wheeler, M. A., Takenaka, M. C., and Quintana, F. J. (2017). Role of AHR and HIF-1alpha in Glioblastoma Metabolism. Trends Endocrinol Metab 28, 428-436.
Ganesh, S., Shui, X., Craig, K. P., Koser, M. L., Chopda, G. R., Cyr, W. A., Lai, C., Dudek, H., Wang, W., Brown, B. D., et al. (2018). beta-Catenin mRNA Silencing and MEK Inhibition Display Synergistic Efficacy in Preclinical Tumor Models. Mol Cancer Ther 17, 544-553.
Gay, C. M., Balaji, K., and Byers, L. A. (2017). Giving AXL the axe: targeting AXL in human malignancy. Br J Cancer 116, 415-423.
Horii, A., Nakatsuru, S., Miyoshi, Y., Ichii, S., Nagase, H., Ando, H., Yanagisawa, A., Tsuchiya, E., Kato, Y., and Nakamura, Y. (1992). Frequent somatic mutations of the APC gene in human pancreatic cancer. Cancer Res 52, 6696-6698.
Jin, Y., Nie, D., Li, J., Du, X., Lu, Y., Li, Y., Liu, C., Zhou, J., and Pan, J. (2017). Gas6/AXL Signaling Regulates Self-Renewal of Chronic Myelogenous Leukemia Stem Cells by Stabilizing beta-Catenin. Clin Cancer Res 23, 2842-2855.
Kariolis, M. S., Miao, Y. R., Diep, A., Nash, S. E., Olcina, M. M., Jiang, D., Jones, D. S., 2nd, Kapur, S., Mathews, II, Koong, A. C., et al. (2017). Inhibition of the GAS6/AXL pathway augments the efficacy of chemotherapies. J Clin Invest 127, 183-198.
Kawajiri, K., Kobayashi, Y., Ohtake, F., Ikuta, T., Matsushima, Y., Mimura, J., Pettersson, S., Pollenz, R. S., Sakaki, T., Hirokawa, T., et al. (2009). Aryl hydrocarbon receptor suppresses intestinal carcinogenesis in ApcMin/+ mice with natural ligands. Proc Natl Acad Sci USA 106, 13481-13486.
Liu, Y., and Cao, X. (2015). The origin and function of tumor-associated macrophages. Cell Mol Immunol 12, 1-4.
Loges, S., Schmidt, T., Tjwa, M., van Geyte, K., Lievens, D., Lutgens, E., Vanhoutte, D., Borgel, D., Plaisance, S., Hoylaerts, M., et al. (2010). Malignant cells fuel tumor growth by educating infiltrating leukocytes to produce the mitogen Gas6. Blood 115, 2264-2273.
Ohgaki, H., Kros, J. M., Okamoto, Y., Gaspert, A., Huang, H., and Kurrer, M. O. (2004). APC mutations are infrequent but present in human lung cancer. Cancer Lett 207, 197-203.
Opitz, C. A., Litzenburger, U. M., Sahm, F., Ott, M., Tritschler, I., Trump, S., Schumacher, T., Jestaedt, L., Schrenk, D., Weller, M., et al. (2011). An endogenous tumour-promoting ligand of the human aryl hydrocarbon receptor. Nature 478, 197-203.
Opitz, C. A., Somarribas Patterson, L. F., Mohapatra, S. R., Dewi, D. L., Sadik, A., Platten, M., and Trump, S. (2020). The therapeutic potential of targeting tryptophan catabolism in cancer. Br J Cancer 122, 30-44.
Orchard, G., Martin, J. G., Vogelstein, R. J., and Etienne-Cummings, R. (2013). Fast neuromimetic object recognition using FPGA outperforms GPU implementations. IEEE Trans Neural Netw Learn Syst 24, 1239-1252.
Ott, M., Litzenburger, U. M., Rauschenbach, K. J., Bunse, L., Ochs, K., Sahm, F., Pusch, S., Opitz, C. A., Blaes, J., von Deimling, A., et al. (2015). Suppression of TDO-mediated tryptophan catabolism in glioblastoma cells by a steroid-responsive FKBP52-dependent pathway. Glia 63, 78-90.
Pate, K. T., Stringari, C., Sprowl-Tanio, S., Wang, K., TeSlaa, T., Hoverter, N. P., McQuade, M. M., Garner, C., Digman, M. A., Teitell, M. A., et al. (2014). Wnt signaling directs a metabolic program of glycolysis and angiogenesis in colon cancer. EMBO J 33, 1454-1473.
Pathria, P., Louis, T. L., and Varner, J. A. (2019). Targeting Tumor-Associated Macrophages in Cancer. Trends Immunol 40, 310-327.
Peranzoni, E., and Donnadieu, E. (2019). Improving efficacy of cancer immunotherapy through targeting of macrophages. Hum Vaccin Immunother 15, 189-192.
Pilotte, L., Larrieu, P., Stroobant, V., Colau, D., Dolusic, E., Frederick, R., De Plaen, E., Uyttenhove, C., Wouters, J., Masereel, B., et al. (2012). Reversal of tumoral immune resistance by inhibition of tryptophan 2,3-dioxygenase. Proc Natl Acad Sci USA 109, 2497-2502.
Puccetti, P. (2014). On the Non-Redundant Roles of TDO2 and IDO1. Front Immunol 5, 522. Quintana, F. J., Basso, A. S., Iglesias, A. H., Kom, T., Farez, M. F., Bettelli, E., Caccamo, M., Oukka, M., and Weiner, H. L. (2008). Control of T(reg) and T(H)17 cell differentiation by the aryl hydrocarbon receptor. Nature 453, 65-71.
Sadahiro, H., Kang, K. D., Gibson, J. T., Minata, M., Yu, H., Shi, J., Chhipa, R., Chen, Z., Lu, S., Simoni, Y., et al. (2018). Activation of the Receptor Tyrosine Kinase AXL Regulates the Immune Microenvironment in Glioblastoma. Cancer Res 78, 3002-3013.
Stange, J., and Veldhoen, M. (2013). The aryl hydrocarbon receptor in innate T cell immunity. Semin Immunopathol 35, 645-655.
Su, L. K., Kinzler, K. W., Vogelstein, B., Preisinger, A. C., Moser, A. R., Luongo, C., Gould, K. A., and Dove, W. F. (1992). Multiple intestinal neoplasia caused by a mutation in the murine homolog of the APC gene. Science 256, 668-670.
Takenaka, M. C., Gabriely, G., Rothhammer, V., Mascanfroni, I. D., Wheeler, M. A., Chao, C. C., Gutierrez-Vazquez, C., Kenison, J., Tjon, E. C., Barroso, A., et al. (2019). Control of tumor-associated macrophages and T cells in glioblastoma via AHR and CD39. Nat Neurosci 22, 729-740.
van Baren, N., and Van den Eynde, B. J. (2015). Tryptophan-degrading enzymes in tumoral immune resistance. Front Immunol 6, 34.
Van der Flier, L. G., Sabates-Bellver, J., Oving, I., Haegebarth, A., De Palo, M., Anti, M., Van Gijn, M. E., Suijkerbuijk, S., Van de Wetering, M., Marra, G., et al. (2007). The Intestinal Wnt/TCF Signature. Gastroenterology 132, 628-632.
Zhao, D., Lu, X., Wang, G., Lan, Z., Liao, W., Li, J., Liang, X., Chen, J. R., Shah, S., Shang, X., et al. (2017). Synthetic essentiality of chromatin remodelling factor CHD1 in PTEN-deficient cancer. Nature 542, 484-488.

1. (canceled)

2. A method for treating a subject for an APC-deficient cancer and/or constitutively active WNT signaling, the method comprising providing to the subject a pharmaceutical composition comprising an effective amount of an inhibitor of (a) tryptophan 2,3-dioxygenase (TDO2) or (b) a cytokine activated by TDO2 activity or a receptor thereof.

3. (canceled)

4. The method of claim 2, wherein the APC-deficient cancer is a cancer that comprises cancerous cells having an APC mutation.

5-6. (canceled)

7. The method of claim 2, wherein inhibitor is an inhibitor of the expression or activity of TDO2.

8-9. (canceled)

10. The method of claim 2, wherein the inhibitor is an siRNA, an shRNA, an antisense oligonucleotide, a small molecule inhibitor, an antibody, or an antibody-like molecule.

11. (canceled)

12. The method of claim 10, wherein the inhibitor is PF06845102/EOS200809, 680C91, LM10, HTI-1090, DN1406131, RG70099, EPL-1410, CB548, CMG017, or a derivative thereof.

13. The method of claim 2, wherein the inhibitor is an inhibitor of a cytokine activated by TDO2 activity.

14. The method of claim 13, wherein the cytokine is CXCL5, CXCL7, CSF3, CXCR2, CXCL2, CXCL10, CCL2, CXCL1, CXCR1/2, or CXCR5.

15-21. (canceled)

22. The method of claim 2, wherein the cancer has an increased expression of TDO2 relative to a control or reference sample and wherein the control or reference sample is a biological sample from a healthy subject.

23. (canceled)

24. The method of claim 2, wherein the cancer comprises constitutively active WNT signaling.

25. The method of claim 2, wherein the cancer is colorectal cancer, breast cancer, prostate cancer, lung cancer, head and neck squamous cell carcinoma, or sarcoma.

26-36. (canceled)

37. The method of claim 2, further comprising providing to the subject a cancer immunotherapy, wherein the immunotherapy comprises an antibody therapy, a cellular therapy, or a checkpoint inhibitor therapy.

38-39. (canceled)

40. The method of claim 2, further comprising providing to the subject an additional therapy, wherein the additional therapy is chemotherapy, radiation therapy, surgery, or a combination thereof.

41-43. (canceled)

44. The method of claim 2, further comprising providing to the subject an inhibitor of indoleamine 2,3-dioxygenase 1 (IDO1) or indoleamine 2,3-dioxygenase 2 (IDO2).

45. The method of claim 2, wherein the subject has been treated with an inhibitor of IDO1 or IDO2.

46-50. (canceled)

51. A method for identifying a cancer as being sensitive to TDO2 inhibition, the method comprising:
(a) obtaining cancer cells from a biological sample from a subject;
(b) detecting a deficiency in an APC gene or detecting a WNT activating mutation in the cancer cells; and
(c) identifying the cancer as being sensitive to TDO2 inhibition based on (b).

52-72. (canceled)

73. The method of claim 2, wherein the constitutively active WNT signaling comprises a constitutively active β-catenin.

74. The method of claim 13, wherein the cancer comprises a constitutively active β-catenin protein.

75. A method for treating cancer in a subject comprising determining whether the cancer has an APC mutation and:
(a) if the cancer has an APC mutation, providing to the subject an effective amount of an inhibitor of (ii) TDO2 or (ii) a cytokine activated by TDO2 activity; and
(b) if the cancer does not have an APC mutation, providing to the subject an effective amount of an alternate therapy.

76-81. (canceled)

82. The method of claim 45, wherein the subject has been determined to be resistant to the inhibitor of IDO1 or IDO2.


SEQUENCE LISTING (1 sequence)

SEQ ID NO: 1 - protein, 406 aa, Homo sapiens
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD